(19) United States
(12) Patent Application Publication
(10) Pub. No.: US 2026/0340649 A1
(43) Pub. Date: Oct. 29, 2026

(54) CARBON DIOXIDE EMISSIONS CALCULATION SYSTEM AND METHOD

(71) Applicant: KABUSHIKI KAISHA KOBE SEIKO SHO (KOBE STEEL, LTD.), Hyogo (JP)

(72) Inventor: Toyohiro UMEDA, Kobe-shi, Hyogo (JP)

(73) Assignee: KABUSHIKI KAISHA KOBE SEIKO SHO (KOBE STEEL, LTD.), Hyogo (JP)

(21) Appl. No.: 19/117,858

(22) PCT Filed: Sep. 27, 2023
(86) PCT No.: PCT/JP2023/035160

§ 371 Date: Apr. 2, 2025

(30) Foreign Application Priority Data
Dec. 2, 2022 (JP) 2022-193236
Jan. 16, 2023 (JP) 2023-004652

(51) Int. Cl. G06Q 30/018; G01N 33/00

(52) U.S. Cl. CPC G06Q 30/018; G01N 33/004

(57) ABSTRACT

The present invention is a carbon dioxide emission amount calculation system that calculates CO₂ emission amounts about a target product of a target type in a plant configured to produce a certain product of a plurality of types through a plurality of steps. The system includes: calculating, on the basis of a total production load amount required in production in each of main facilities respectively associated with the steps in a predetermined period and a total CO₂ emission amount with respect to the total production load amount, a unit CO₂ emission amount per unit about the main facility; and calculating a step CO₂ emission amount about the associated step executed by the main facility in connection with the production of the target product of the target type on the basis of the unit CO₂ emission amount and the total production load amount about the main facility in connection with the production of the target product of the target type.

### TECHNICAL FIELD

The present invention relates to a carbon dioxide emission amount calculation system and a carbon dioxide emission amount calculation method to calculate an emission amount of carbon dioxide emitted or discharged in relation to a product of a predetermined type produced in a predetermined period.

### BACKGROUND ART

A carbon dioxide emission or discharge amount has recently drawn attention from the viewpoint of global environmental conservation. For instance, Patent Literature 1 describes a technology concerning such a carbon dioxide emission amount. Patent Literature 1 discloses a carbon traceability management system including: a utility management part that records and manages electric power received from outside, received cooling or warming energy amount, a private power generator actual operation performance, and a cooling and warming facility actual operation performance; a material management part that records and manages a material and a raw material actual use performance; a facility management part that manages and records a consumed energy amount of a facility during a period of production; a production execution management part that manages an actual production performance for a product; an emission amount calculation part that calculates a greenhouse effect gas emission amount from the actual performances recorded and managed respectively by the utility management part, the material management part, the facility management part, and the production execution management part; and an emission amount allotment part that allots the greenhouse effect gas emission amount calculated by the emission amount calculation part to production lots each representing an amount per product unit. A boiler, a power receipt facility, a private power generator, and a water supply are each classified as a primary powering facility. A powering facility that is activated by receiving supply of energy from the primary powering facility is classified as a secondary powering facility, and a non-powering facility that is activated by receiving supply of energy from the primary powering facility and the secondary powering facility is classified as a tertiary facility. The emission amount allotment part allots the greenhouse effect gas emission amount associated with each production lot, depending on a usage status of the tertiary facility associated with the production lot and on a usage status of the secondary facility associated with the production lot, and includes an emission amount management section that manages the greenhouse effect gas emission amount allotted to the production lot as data peculiar to the production lot.

The carbon traceability management system disclosed in Patent Literature 1 enables calculation of the greenhouse effect gas emission amount per production lot representing an amount per product unit. Unfortunately, the carbon traceability management system disclosed in Patent Literature 1 needs to manage various kinds of data, which are required for calculation of the greenhouse effect gas emission amount by the system, in association with production lots in a time sequence. The system is thus unavailable without such management based on the time sequential association.

### CITATION LIST

### Patent Literature

Patent Literature 1: Japanese Patent Publication No. 5097728

### SUMMARY OF INVENTION

The present invention has been accomplished in view of the circumstances described above, and has an object of providing a carbon dioxide emission amount calculation system and a carbon dioxide emission amount calculation method each of which enables calculation of a carbon dioxide emission amount per target product of each target type even without the aforementioned association.

A carbon dioxide emission amount calculation system and a carbon dioxide emission amount calculation method according to the present invention aim at calculating CO₂ emission amounts about a target product of a target type in a plant configured to produce a certain product of a plurality of types through a plurality of steps. The system and the method include: calculating, on the basis of a total production load amount required in a main facility associated with each of the steps in a predetermined period in connection with production and a total CO₂ emission amount with respect to the total production load amount, a unit CO₂ emission amount per unit about the main facility; and calculating, on the basis of the unit CO₂ emission amount and the total production load amount about the main facility in connection with production of the target product of the target type, a step CO₂ emission amount about the step executed by the main facility in connection with the production of the target product of the target type.

The foregoing, and the object, features, and advantages of the present invention will be further clarified by the following detailed description and the accompanying drawings.

### BRIEF DESCRIPTION OF DRAWINGS

FIG. 1 is a diagram illustrating a configuration of a carbon dioxide emission amount calculation system in an embodiment.

FIG. 2 includes illustrations of example production step information tables.

FIG. 3 illustrates an example product actual production performance information table.

FIG. 4 illustrates an example facility-related actual performance information table.

FIG. 5 illustrates an example remaining emission source-related actual performance information table.

FIG. 6 includes illustrations of example unit emission amount information tables.

FIG. 7 illustrates an example auxiliary facility correlation information matrix.

FIG. 8 illustrates an example table for explanation of a way of calculating a total step CO2 emission amount about a step executed by a main facility and a way of calculating a total auxiliary facility CO₂ emission amount about an auxiliary facility.

FIG. 9 includes illustrations of example tables for explanation of a way of calculating a CO2 emission allotment amount about a main facility that utilizes an auxiliary facility.

FIG. 10 illustrates an example table for explanation of a way of calculating a second unit CO2 emission amount per unit about a main facility.

FIG. 11 illustrates an example table for explanation of a way of calculating a fourth unit CO₂ emission amount per product unit about a remaining emission source.

FIG. 12 illustrates an example output of calculation results.

FIG. 13 illustrates another example output of calculation results.

FIG. 14 is a flowchart showing an operation of the carbon dioxide emission amount calculation system.

FIG. 15 includes illustrations for explanation of a first addition way in a modified embodiment.

FIG. 16 includes illustrations for explanation of a second addition way in a modified embodiment.

FIG. 17 includes illustrations for explanation of a third addition way in a modified embodiment.

FIG. 18 includes illustrations for explanation of a fourth addition way in a modified embodiment.

### DESCRIPTION OF EMBODIMENTS

Hereinafter, one or more embodiments of the present invention will be described with reference to the accompanying drawings. However, the present invention should not be limited to the disclosed embodiments. Elements denoted by the same reference numerals in the drawings have the same configuration and, therefore, repeated descriptions will be appropriately omitted. In the present specification, elements are denoted by a same reference numeral when being referred to collectively, and are denoted by a same reference numeral accompanied by a different respective reference character when being referred to individually.

A carbon dioxide emission amount calculation system in an embodiment calculates carbon dioxide (CO₂) emission or discharge amounts about a target product of a target type in a plant configured to produce a certain product of a plurality of types through a plurality of steps. The carbon dioxide emission amount calculation system includes a raw material emission amount calculation part, a main facility unit emission amount calculation part, and a step emission amount calculation part. The raw material emission amount calculation part calculates, on the basis of a given (predetermined, or prepared in advance) first unit CO₂ emission amount per unit about a raw material in connection with production of the target product of the target type and a total amount of the raw material used for the target product of the target type produced in a predetermined period, a total raw material CO₂ emission amount about the raw material in connection with the production of the target product of the target type. The main facility unit emission amount calculation part calculates, for each of a plurality of main facilities respectively associated with the steps in the production of the target product of the target type, a second unit CO₂ emission amount per unit about each of the main facilities on the basis of a total production load amount required in the main facility associated with the step to execute the step in the production in the predetermined period and a total CO₂ emission amount about the main facility with respect to the total production load amount. The step emission amount calculation part calculates, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated by the main facility unit emission amount calculation part and the total production load amount required in the main facility in the predetermined period in connection with the production of the target product of the target type.

Hereinafter, the carbon dioxide emission amount calculation system and a carbon dioxide emission amount calculation method for the system will be described in more detail. Although a factory or plant configured to produce a rolled steel plate of various kinds will be described as an example here, any other factory or plant configured to produce a certain product of a plurality of types through a plurality of steps is adoptable. The carbon dioxide emission amount calculation system may include: an input and output terminal device that inputs and outputs data; one or more calculation processing devices (e.g., servers) that execute various kinds of arithmetic processing or calculations; and one or more database devices that store or manage various kinds of data, the devices being communicably connected to one another. The system may be established by integrating at least a part of: the input and output terminal device; the one or more calculation processing devices; and the one or more database devices, in such a manner as to be communicably connected to the remaining one or more devices. Here, the carbon dioxide emission amount calculation system will be described as a representative of a carbon dioxide emission amount calculation apparatus integrally including all the devices.

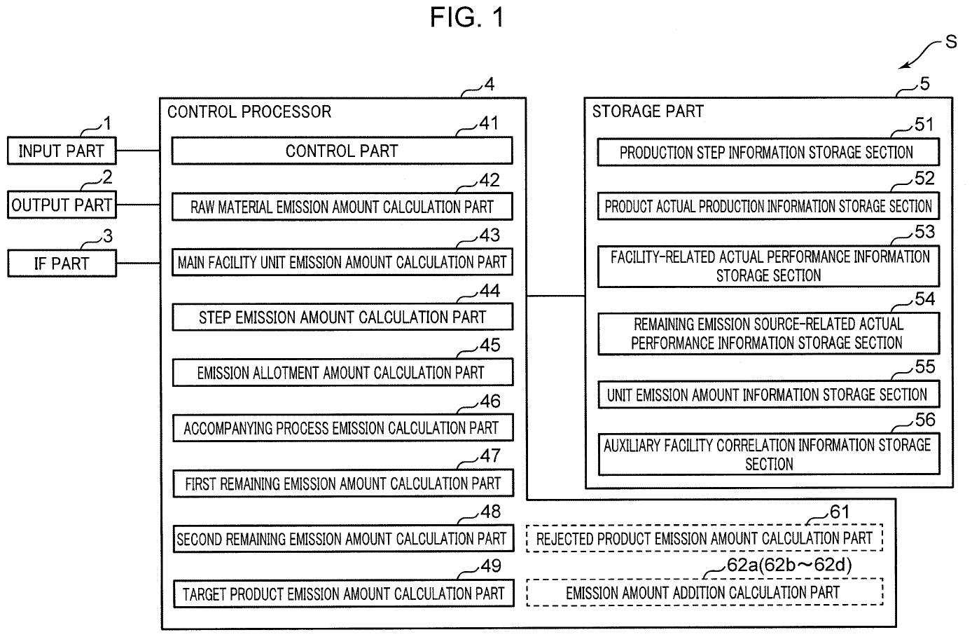

FIG. 1 is a diagram illustrating a configuration of a carbon dioxide emission amount calculation system (a carbon dioxide emission amount calculation apparatus serving as one example of the system) in the embodiment.

Such a carbon dioxide emission amount calculation system (the carbon dioxide emission amount calculation apparatus serving as one example of the system) S in the embodiment includes, for example, an input part 1, an output part 2, an interface part (IF part) 3, a control processor 4, and a storage part 5 as illustrated in FIG. 1.

The input part 1 is connected to the control processor 4 and inputs, into the carbon dioxide emission amount calculation apparatus S, various commands, such as a command to instruct start of calculation of a CO₂ emission amount, and various data, such as a production step for a product and an actual production performance for a product, the data being required to activate the carbon dioxide emission amount calculation apparatus S. Examples of the input part include a plurality of input switches to which predetermined functions are assigned, a keyboard, and a mouse. The output part 2 is connected to the control processor 4, and outputs the commands and data input from the input part 1, under the control by the control processor 4. Examples of the output part include a display device, such as a cathode ray tube (CRT) display, a liquid crystal display (LCD), and an organic electroluminescence (EL) display, and a printing device, such as a printer.

Alternatively, the input part 1 and the output part 2 may constitute a touch screen. In the configuration of the touch screen, the input part 1 serves as, for example, a position input device of a resistive film type or an electrostatic capacitive type that detects and inputs an operation position, and the output part 2 serves as a display device. This touch screen is provided with the position input device on a display screen surface of the display device. The display device displays one or more input content candidates that can be input into the display device. When a user touches a display position where an input content desired to be input is displayed, the position input device detects the position. The display content displayed at the detected position is input into the carbon dioxide emission amount calculation apparatus S as a user's operation input content. Such a touch screen allows the user to understand an input operation intuitively and easily. This results in making the carbon dioxide emission amount calculation apparatus S user-friendly.

For instance, the IF part 3 is a circuit that is connected to the control processor 4 and inputs and outputs data into and from an external device under the control by the control processor 4, and is, for example, an interface circuit of RS-232C which is a serial communication way, an interface circuit using the Bluetooth (registered trademark) standard, or an interface circuit using a Universal Serial Bus (USB) standard. Alternatively, the IF part 3 may be a communication interface circuit that receives and sends a communication signal from and to an external device, e.g., a data communication card or a communication interface circuit conforming to the Institute of Electrical and Electronics Engineers (IEEE) 802.11 standard.

The storage part 5 is a circuit that is connected to the control processor 4 and stores various predetermined programs and various kinds of predetermined data under the control by the control processor 4. For instance, the various predetermined programs may be recorded in a recording medium, e.g., a CD-ROM or a DVD-R, and may be input into and stored in the storage part 5 from the recording medium via a drive device and the IF part 3.

The various predetermined programs include, for example, control processing programs. For instance, the control processing programs include: a control program; a raw material emission amount calculation program; a main facility unit emission amount calculation program; a step emission amount calculation program; an emission allotment amount calculation program; an accompanying process emission amount calculation program; a first remaining emission amount calculation program; a second remaining emission amount calculation program; and a target product emission amount calculation program. The control program is intended for execution of control of the parts 1 to 3 and 5 of the carbon dioxide emission amount calculation apparatus S in accordance with operability of these parts. The raw material emission amount calculation program is a program for calculating, on the basis of a given first unit CO₂ emission amount per unit about a raw material in connection with production of the target product of the target type and a total amount of the raw material used for the target product of the target type produced in a predetermined period, a total raw material CO₂ emission amount about the raw material in connection with the production of the target product of the target type. The main facility unit emission amount calculation program is a program for calculating, for each of a plurality of main facilities respectively associated with the steps in the production of the target product of the target type, a second unit CO₂ emission amount per unit about each of the main facilities on the basis of a total production load amount required in the main facility associated with the step to execute the step in the production in the predetermined period and a total CO₂ emission amount about the main facility with respect to the total production load amount. The step emission amount calculation program is a program for calculating, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated with the main facility unit emission amount calculation program and the total production load amount required in the main facility in the predetermined period in connection with the production of the target product of the target type. The emission allotment amount calculation program is a program for calculating, concerning an auxiliary facility indirectly involved in the production of the product via a specific main facility and being adoptable for a plurality of facilities, a CO₂ emission allotment amount about a certain facility that utilizes the auxiliary facility by allotting a total auxiliary facility CO₂ emission amount about the auxiliary facility in the predetermined period to each of the facilities that utilizes the auxiliary facility at a predetermined proportion. The accompanying process emission amount calculation program is a program for calculating, on the basis of a given third unit CO₂ emission amount per unit about a predetermined process executed to the product after the production of the product to delivery of the product to a delivery destination and a total amount of the target product of the target type produced in the plant in the predetermined period, a total accompanying process CO₂ emission amount about the predetermined process to the target product of the target type. The first remaining emission amount calculation program is a program for calculating a fourth unit CO₂ emission amount per product unit about the remaining emission source on the basis of a total remaining CO₂ emission amount about the remaining emission source in the predetermined period and a total amount of the product produced in the plant in the predetermined period. The second remaining emission amount calculation program is a program for calculating a total second remaining CO₂ emission amount about the remaining emission source in connection with the production of the target product of the target type on the basis of the fourth unit CO₂ emission amount per product unit about the remaining emission source calculated with the first remaining emission amount calculation program and a total amount of the target product of the target type produced in the predetermined period. The target product emission amount calculation program is a program for calculating, as a total target product CO₂ emission amount about the target product of the target type, a sum of: a total of respective step CO₂ emission amounts calculated with the step emission amount calculation program about the steps executed by the main facilities for each of the main facilities respectively associated with the steps in the production of the target product of the target type; and the total raw material CO₂ emission amount about the raw material calculated with the raw material emission amount calculation program in connection with the production of the target product of the target type.

Examples of the various kinds of predetermined data include data necessary to execute each program, such as production step information, product actual production performance information, facility-related actual performance information, remaining emission source-related actual performance information, unit emission amount information, auxiliary facility correlation information, various calculation results in progress of calculations, and final calculation results.

The storage part 5 includes, for example, a read only memory (ROM) that is a nonvolatile storage element, an electrically erasable programmable read only memory (EEPROM) that is a rewritable nonvolatile storage element, or other storage element. The storage part 5 includes a random access memory (RAM) serving as a so-called working memory of the control processor 4 that stores data and the like generated in execution of the predetermined programs. The storage part 5 may include a hard disk device having a relatively large storage capacity.

The storage part 5 includes a production step information storage section 51 to store the production step information, a product actual production performance information storage section 52 to store the product actual production performance information, a facility-related actual performance information storage section 53 to store the facility-related actual performance information, a remaining emission source-related actual performance information storage section 54 to store the remaining emission source-related actual performance information, a unit emission amount information storage section 55 to store the unit emission amount information, and an auxiliary facility correlation information storage section 56 to store the auxiliary facility correlation information.

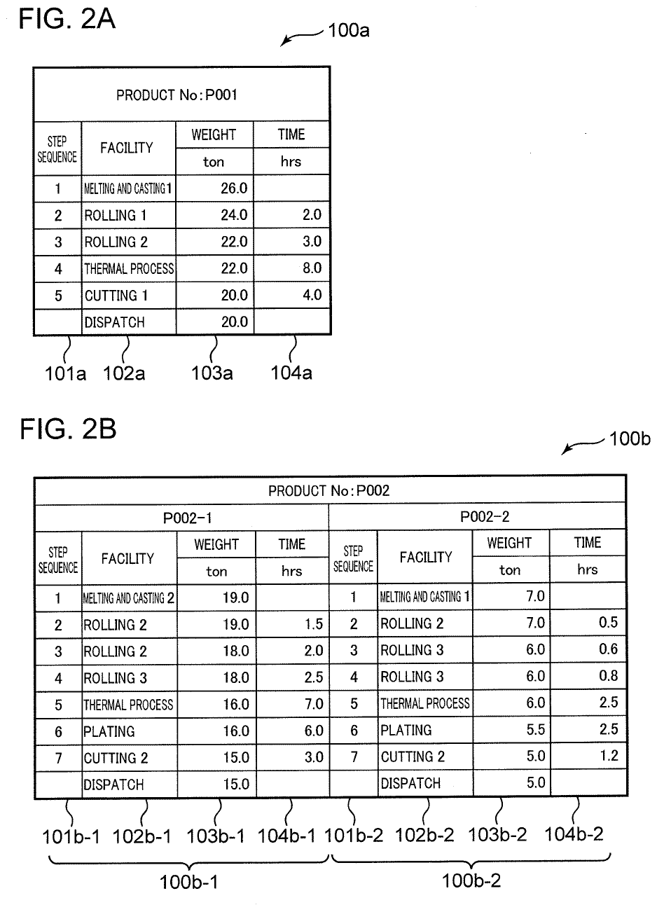

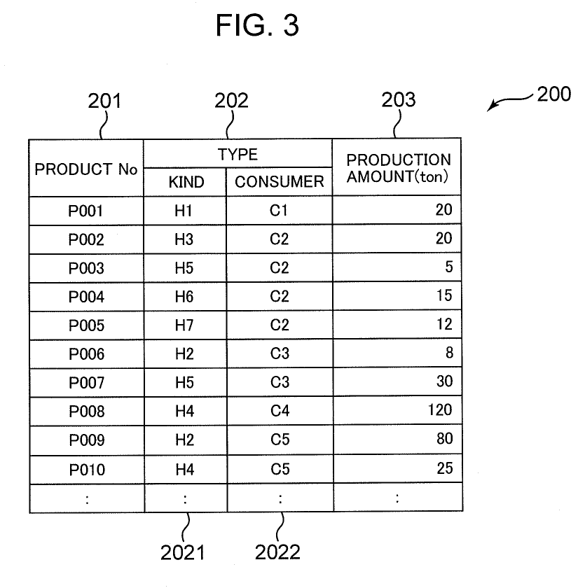

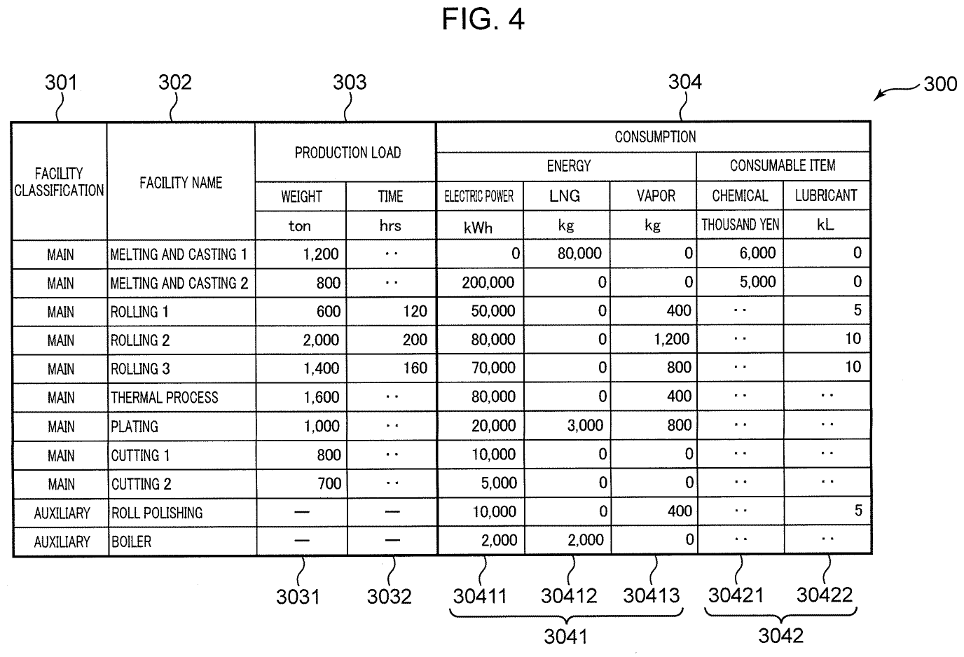

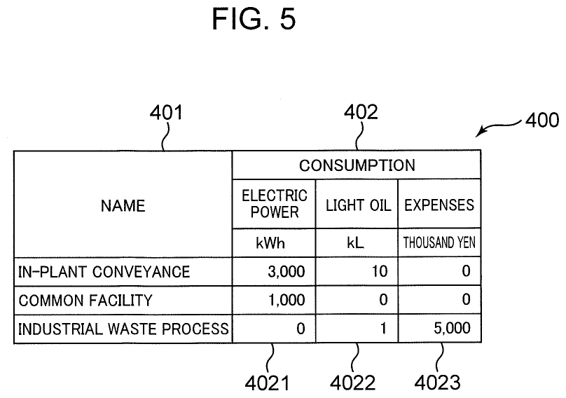

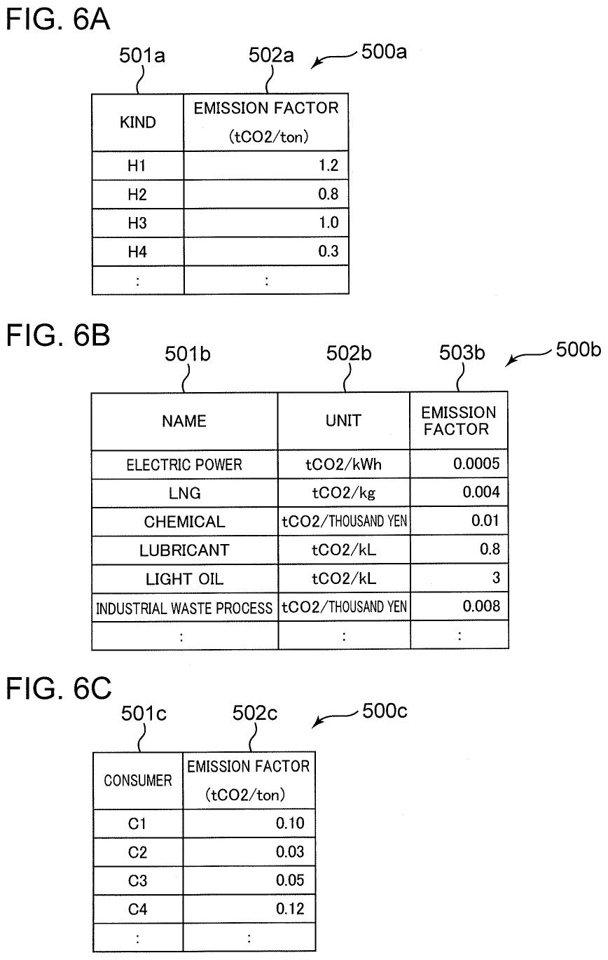

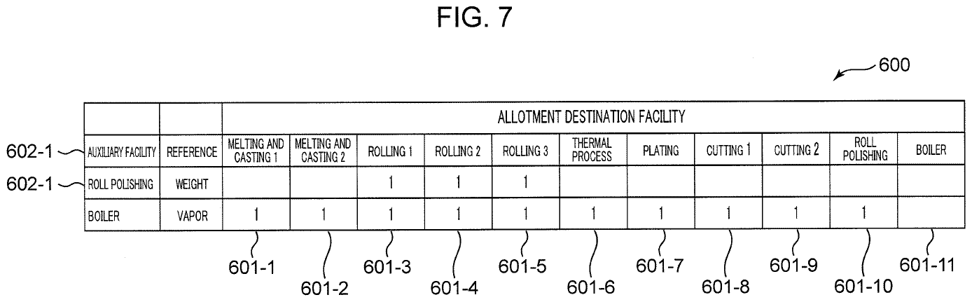

FIG. 2 includes illustrations of example production step information tables. FIG. 2A illustrates a production step information table for a product with the product number “P001”. FIG. 2B illustrates a production step information table for a product with the product number “P002”. Each product number represents an identifier to specify and identify each product. FIG. 3 illustrates an example product actual production performance information table. FIG. 4 illustrates an example facility-related actual performance information table. FIG. 5 illustrates an example remaining emission source-related actual performance information table. FIG. 6 includes illustrations of example unit emission amount information tables. FIG. 6A illustrates a unit emission amount information table showing a first unit CO₂ emission amount per unit about a raw material. FIG. 6B illustrates a unit emission amount information table showing each unit CO₂ emission amount per unit about each main facility, each auxiliary facility, and each remaining emission source. FIG. 6C illustrates a unit emission amount information table showing a third unit CO₂ emission amount per unit about a predetermined process. FIG. 7 illustrates an example auxiliary facility correlation information matrix.

The production step information storage section 51 stores the production step information. The production step information includes information or data indicating a production step for a product per type of product.

In the embodiment, the production step information storage section 51 stores the production step information in a table format. As illustrated in FIG. 2, for instance, a production step information table 100 (100a, 100b) having registration of the production step information includes a step sequence field 101 (101a, 101b, i.e., 101b-1 and 101b-2) having registration of steps in a sequence, and a facility field 102 (102a, 102b, i.e., 102b-1 and 102b-2) having registration of main facilities used in execution of the steps in the sequence registered in the step sequence field 101, the table having a record for each step. Each main facility indicates a facility associated with a specific step to execute the step, the facility being appropriately determined suitably for a product to be produced in a plant. In the embodiment, the plant is configured to produce a rolled steel plate of various kinds as described above. Thus, examples of the main facility include a welding and casting facility, a rolling facility, a thermal process facility, and a cutting facility. One or more main facilities of the same type may be provided.

In the embodiment, the production step information table 100 is defined to have registrations of various actual performances in respective steps for the product of the type. The production step information table 100 further includes a weight field 103 (103a, 103b, i.e., 103b-1, 103b-2) and a time field 104 (104a, 104b, i.e., 104b-1, 104b-2), the weight field having registration of a total produced object amount (an actual performance weight) for the product of the type produced in a predetermined period in each main facility to execute each step in the sequence registered in the step sequence field 101, the time field having registration of a total usage time (a total operation time, an actual performance time) required in the main facility to execute the step in the sequence registered in the step sequence field 101 in the production of the product of the type in the predetermined time period. The table further has a record having registration of a dispatch amount (an actual dispatch amount, a production amount or an actual production amount) of the product of the type in the weight field 103. Here, in the embodiment, an actual performance value registered in the weight field 103 in the first step also represents a total amount of the raw material used in the production of the product of the type in the predetermined period. In the embodiment, a production load of each main facility is expressed with at least one of a production amount of each produced object produced in the main facility and a usage time of the main facility. In this regard, the weight field 103 has registration of the production load in terms of the production amount of the produced object, and the time field 104 has registration of the production load in terms of the usage time.

FIG. 2 includes illustrations of the production step information tables 100a and 100b respectively for two types of the product, that is, for the product with the product number “P001” and the product with the product number “P002”. It is noted here that such a production step information table is prepared in advance for each type of product, and stored in the production step information storage section 51.

In a case where a product of the same type is producible through different steps, the production step information table 100 is created for each different step and stored in the production step information storage section 51. For instance, as illustrated in FIG. 2B, the product with the product number “P002” is producible in a first production step pattern and a second production step pattern which is different from the first production step pattern. The first production step pattern includes steps to be executed in a sequence of: a first step by a main facility with a facility name “melting and casting 2”; a second step by a main facility with a facility name “rolling 2”; a third step by the main facility with the facility name “rolling 2”; a fourth step by a main facility with a facility name “rolling 3”; a fifth step by a main facility with a facility name “thermal process”; a sixth step by a main facility with a facility name “plating”; and a seventh step by a main facility with a facility name “cutting 2”. The second production step pattern includes steps to be executed in a sequence of: a first step by a main facility with a facility name “melting and casting 1”; a second step by the main facility with the facility name “rolling 2”; a third step by the main facility with the facility name “rolling 3”; a fourth step by the main facility with the facility name “rolling 3”; a fifth step by the main facility with the facility name “thermal process”; a sixth step by the main facility with the facility name “plating”; and a seventh step by the main facility with the facility name “cutting 2”. The production step information table 102b for the product with the product number “P002” includes a table 102b-1 having registration of production step information about the first production step pattern and a table 102b-2 having registration of production step information about the second production step pattern.

The product actual production performance information storage section 52 stores the product actual production performance information. The product actual production performance information includes information or data indicating a production amount of a product per type of product in a predetermined period. Examples of the predetermined period include ten days, one month, and a half year to be appropriately preset by a user or operator.

In the embodiment, the product actual production performance information storage section 52 stores the product actual production performance information in a table format. For instance, as illustrated in FIG. 3, a product actual production performance information table 200 having registration of the product actual production performance information includes a product number field 201 having registration of a product number, a type field 202 having registration of a type of a product with the product number registered in the product number field 201, and a production amount field 203 having registration of a production amount of the product with the product number registered in the product number field 201, and the table has a record per product number or type of product. The product number represents an identifier to specify a type of product and identify the type of product. In the embodiment, for example, such a type of product is classified depending on a specification of product. Thus, each product number is also given for each type of product or each specification of product. In the embodiment, the specification of product is classified depending on a kind, and a consumer or an orderer. Accordingly, at receiving of a plurality of orders at different times for the same kind from the same consumer, the same product number is given to these orders and products produced in response to the orders are collected to one group. From this perspective, a target product of a target type may be defined to be one product or to include a plurality of products. The “kind” here represents a name of a steel plate given and classified depending on a composition of the steel plate. The “kind” may result from classification further based on perspectives of strength of a product and a dimension of the product each influenced by a production step. In this respect, in the embodiment, the type field 202 includes a kind field or kind sub-field 2021 having registration of a kind, and a consumer field or consumer sub-field 2022 having registration of a consumer.

A sum of respective production amounts registered in the production amount field 203 in the records is calculated to calculate a total amount of the product of each type produced in the plant in the predetermined period. The total amount reaches 1,500 [ton] in the example in FIG. 3 even without a part of information about the production amounts.

The facility-related actual performance information storage section 53 stores facility-related actual performance information. The facility-related actual performance information includes data indicating, for each of the main facilities and the auxiliary facilities, a total production load amount required in each main facility and each auxiliary facility and a consumption amount of a consumed item accompanied by the operation of the main facility and the auxiliary facility and accompanied by carbon dioxide emission in the predetermined period. The consumption amount includes monetary consumption or expenses as understood from an example to be described later. The auxiliary facility is indirectly involved in the production of the product via the associated main facility and adoptable for a plurality of facilities including such main facilities and auxiliary facilities as to be appropriately determined on the basis of each of the facilities. Examples of the auxiliary facility include a roll polishing facility that polishes a roll of a rolling facility and a boiler facility that supplies vapor to the facilities according to classification.

In the embodiment, the facility-related actual performance information storage section 53 stores the facility-related actual performance information in a table format. As illustrated in FIG. 4, for instance, a facility-related actual performance information table 300 having registration of the facility-related actual performance information includes: a facility name field 302 having registration of a facility name (name of a facility) representing an identifier to specify and identify a facility (each of the main facility and the auxiliary facility); a facility classification field 301 having registration of the facility with the facility name registered in the facility name field 302 as a main facility or an auxiliary facility; a production load field 303 having registration of a total production load amount, in the predetermined period, in the facility with the facility name registered in the facility name field 302; and a consumption field 304 having registration of a consumption amount of a consumed item in the facility with the facility name registered in the facility name field 302. The table has a record for each facility or facility name. The production load field 303 includes sub-fields for a production load per unit, specifically, includes a weight field or weight sub-field 3031 having registration of a total production load amount expressed with a weight [ton] (a total produced object amount in each main facility) about the production load and a time field or time sub-field 3032 having registration of a total production load amount expressed with a usage time [hrs] (a total usage time in the main facility) about the production load. The consumption field 304 includes sub-fields for each type of consumed item, specifically, includes an energy field or energy sub-field 3041 having registration of a consumption amount of energy consumed in operation of a facility and a consumable item field or consumable item sub-field 3042 having registration of a consumption amount of a consumable item used in the facility. The energy field 3041 further includes sub-fields for each type of energy, specifically, includes an electric power field or electric power sub-field 30411 having registration of a consumption amount [kWh] of electric power, an LNG field or LNG sub-field 30412 having registration of a consumption amount [kg] of liquefied natural gas (LNG), and a vapor field or vapor sub-field 30413 having registration of a consumption amount [kg] of vapor. The consumable item field 3042 further includes sub-fields for each type of consumable item, specifically, includes a chemical field or chemical sub-field 30421 having registration of a consumption amount of a chemical and a lubricant field or lubricant sub-field 30422 having registration of a consumption amount of lubricant.

Although the consumed item described above includes energy and consumable items, they are just examples. Other examples of the consumed item may include any item suitable for a type of plant and a type of facility, and include kerosene, refractory, a maintenance work, and industrial water without limitation to the foregoing.

The remaining emission source-related actual performance information storage section 54 stores the remaining emission source-related actual performance information. The remaining emission source includes a remaining CO₂ emission source that emits CO₂ in the plant, except for each main facility, an auxiliary facility in presence of the auxiliary facility, and a predetermined process in presence of the predetermined process. Examples of the remaining CO₂ emission source include: an in-plant conveyor (e.g., a crane, a truck, a forklift, or other vehicle) that conveys articles, such as raw material, a produced object, and a product, in the plant; devices (e.g., a room lighting device, a water heater, an experimental device, and other device) in a common facility (e.g., an office, a welfare facility, an experimental facility, or other facility) that can be shared but is not directly involved in production of the product; and an industrial waste processing facility that processes or copes with industrial wastes in the plant. The predetermined process is executed to a product after production of the product to delivery of the product to a delivery destination. Examples of the predetermined process include: an inspection process of inspecting the product (e.g., an inspection process of inspecting the product whether the product satisfies a dispatch criterion); a wrapping process of wrapping the product; and a transport process of transporting the product from the plant to the delivery destination. The remaining emission source-related actual performance information includes data indicating, for each remaining emission source, a consumption amount of the consumed item accompanied by an operation of the remaining emission source and accompanied by carbon dioxide emission in the predetermined period.

In the embodiment, the remaining emission source-related actual performance information storage section 54 stores the remaining emission source-related actual performance information in a table format. For instance, as illustrated in FIG. 5, a remaining emission source-related actual performance information table 400 having registration of the remaining emission source-related actual performance information includes a name field 401 having registration of a name of a remaining emission source, and a consumption field 402 having registration of a consumption amount of a consumed item about the remaining emission source with the name registered in the name field 401. The table has a record for each remaining emission source. The consumption field 402 includes subfields for each type of consumed item, specifically includes an electric power field or electric power sub-field 4021 having registration of a consumption amount [kWh] of electric power, a light oil field or light oil sub-field 4022 having registration of a consumption amount [kL] of light oil, and an expenses field or expenses sub-field 4023 having registration of expenses [thousand yen]. Concerning an industrial waste processing facility, the expenses field 4023 is provided for a CO₂ emission amount accompanied by a process for industrial wastes in terms of conversion from the expenses in the embodiment.

The unit emission amount information storage section 55 stores the unit emission amount information. The unit emission amount information includes data indicating a unit CO₂ emission amount per unit or emission factor being a factor to convert actual performance values in the product actual production performance information, the facility-related actual performance information, and the remaining emission source-related actual performance information into CO₂ emission amounts.

In the embodiment, the unit emission amount information storage section 55 stores the unit emission amount information in a table format. For instance, as illustrated in FIG. 6A to Fig. 6C, the unit emission amount information table having registration of the unit emission amount information includes three unit emission amount information tables of the first to third unit emission amount information tables 500a to 500c. The number of nit emission amount information tables may be determined appropriately, such as a single collective table, two tables, or four or more tables.

The first unit emission amount information table 500a has registration of a CO₂ emission amount per unit about a raw material, i.e., a first unit CO₂ emission amount per unit. In the embodiment, the raw material is determined depending on a kind of product to be produced with the raw material. Hence, the first unit emission amount information table 500a associates the first unit CO₂ emission amount per unit with such a kind. For instance, as illustrated in FIG. 6A, the first unit emission amount information table 500a includes a kind field 501a having registration of a kind, and an emission factor field 502a having registration of the first unit CO₂ emission amount per unit about the kind registered in the kind field 501a, and the table has a record for each kind. Such association of the first unit CO₂ emission amount per unit with the kind is just an example. The first unit CO₂ emission amount per unit may be associated with a raw material or raw material name without limitation to the association with the kind.

The second unit emission amount information table 500b has registration of a CO₂ emission amount per unit (unit CO₂ emission amount) about each kind, e.g., a CO₂ emission amount per unit about a consumed item (consumed item unit CO₂ emission amount) and a CO₂ emission amount per unit (industrial processing unit CO₂ emission amount) about a process for industrial wastes (industrial waste process) in an industrial waste processing facility in the embodiment. For instance, as illustrated in FIG. 6B, the second unit emission amount information table 500b includes a name field 501b having registration of a name, such as a name of a consumed item, or an industrial waste process, an emission factor field 503b having registration of the unit CO₂ emission amount for the name registered in the name field 501b, and a unit field 502b having registration of a unit of the unit CO₂ emission amount registered in the emission factor field 503b. The table has a record for each name.

The third unit emission amount information table 500c has registration of a CO₂ emission amount per unit about the predetermined process, i.e., third unit CO₂ emission amount per unit, and has registration of a CO₂ emission amount per unit, i.e., transport process unit CO₂ emission amount, about a transport process belonging to the predetermined process in the embodiment. Although the CO₂ emission amount in the transport process is typically determined depending on a transport distance, a total weight of a product to be transported, and a transportation way, the CO₂ emission amount is determined depending on a consumer in the embodiment. Thus, the third unit emission amount information table 500c associates the transport process unit CO₂ emission amount with the consumer. For instance, as illustrated in FIG. 6C, the third unit emission amount information table 500a includes a consumer field 501c having registration of a consumer, and an emission factor field 502c having registration of a transport process unit CO₂ emission amount about the consumer registered in the consumer field 501c, and the table has a record for each consumer. Such association of the transport process unit CO₂ emission amount with the consumer is just an example without limitation to the association.

The auxiliary facility correlation information storage section 56 stores the auxiliary facility correlation information. As described above, an auxiliary facility is involved in the production of the product via an associated main facility and adoptable for a plurality of facilities including such main facilities and other auxiliary facilities for the auxiliary facility. In this respect, the auxiliary facility correlation information includes data indicating a facility that utilizes the auxiliary facility.

The auxiliary facility correlation information storage section 56 stores the auxiliary facility correlation information in a two-dimensional matrix format. For instance, as illustrated in FIG. 7, an auxiliary facility correlation information matrix 600 having registration of the auxiliary facility correlation information includes columns 601-1 to 601-11 respectively having registration of names of facilities that commonly utilize a certain auxiliary facility, and rows 602-1 and 602-2 respectively having registration of names of auxiliary facilities. Each cell where each of the columns 601-1 to 601-11 for the facilities and each of the rows 602-1 and 602-for the auxiliary facilities intersect has registration of a flag “1” to indicate utilization of a specific auxiliary facility by an associated facility, and shows a brank to indicate no utilization of the specific auxiliary facility by any facility. To indicate no utilization of the auxiliary facility by any facility, a flag “0” may be registered to show no utilization. In the example in FIG. 7, the three facilities named “rolling 1”, “rolling 2”, and “rolling 3” commonly utilize a roll polishing facility serving as an auxiliary facility.

The storage part 5 stores, in advance, various actual performance values to calculate CO₂ emission amounts about a target product of a target type before start of calculation of the CO₂emission amounts about the target product of the target type in a predetermined period. The actual performance values may be received through an input from: the input part 1; a storage medium, e.g., a USB memory, an SD card (registered trademark), or other medium, which stores the actual performance value, via the IF part 3; a recording medium, e.g., a CD-R, a DVD-R, or other medium, which records the actual performance value, via a driving device therefor and the IF part 3; a management server that manages the actual performance value via a communication network and the IF part 3; or a main facility or an auxiliary facility via the communication network and the IF part 3 through collection of the values.

In the embodiment, a CO₂ emission amount per unit, i.e., the second unit CO₂ emission amount per unit, about a main facility and a CO₂ emission amount per product unit, i.e., the fourth unit CO₂ emission amount per product unit, about a remaining emission source are calculated and obtained as described below. The rest of various unit CO₂ emission amounts are given and stored in the unit emission amount information storage section 55 in advance. The CO₂ emission amount per unit about the main facility incorporates a CO₂ emission amount about an associated auxiliary facility as described later.

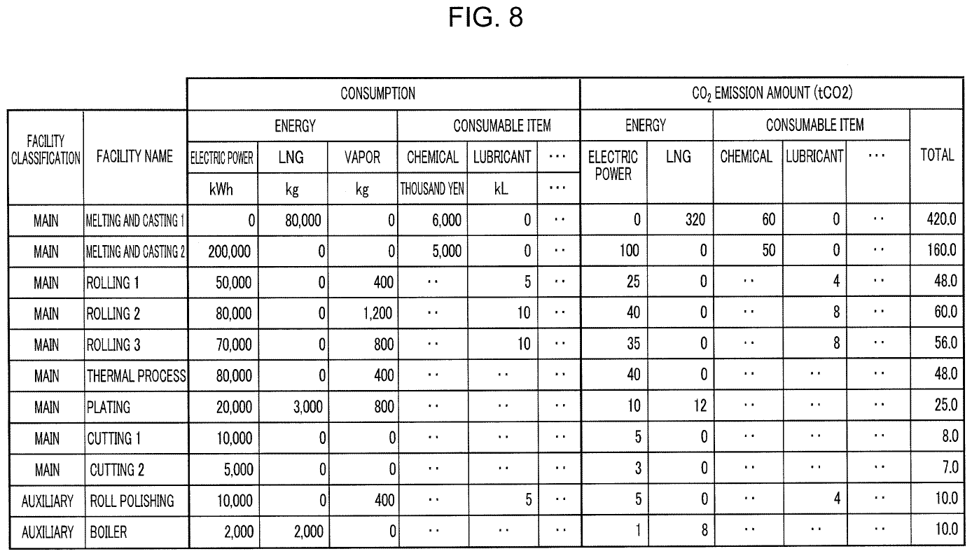

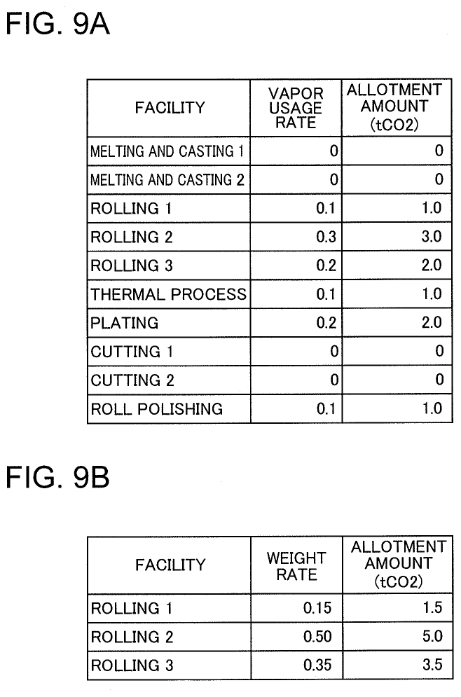

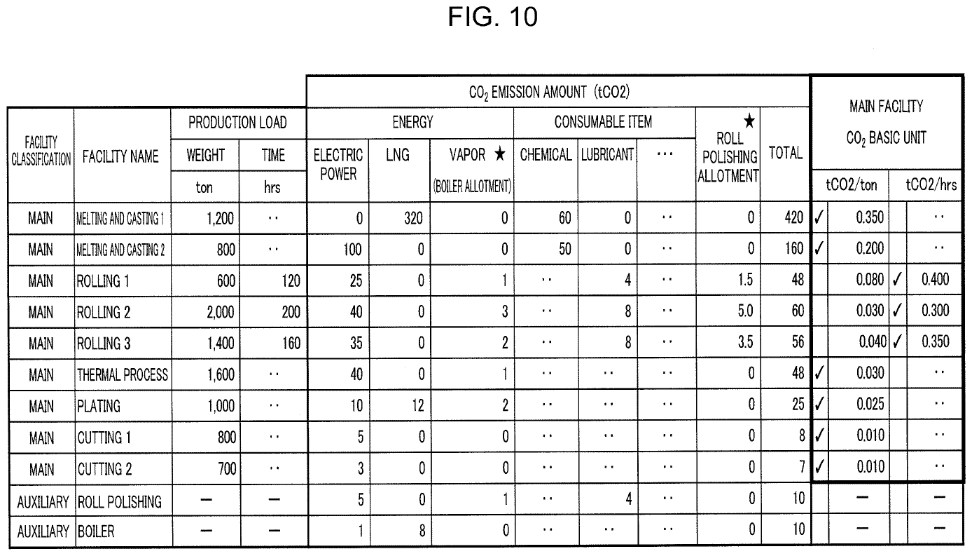

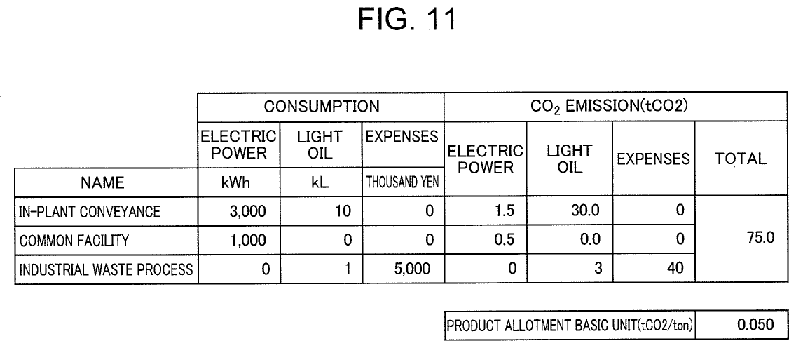

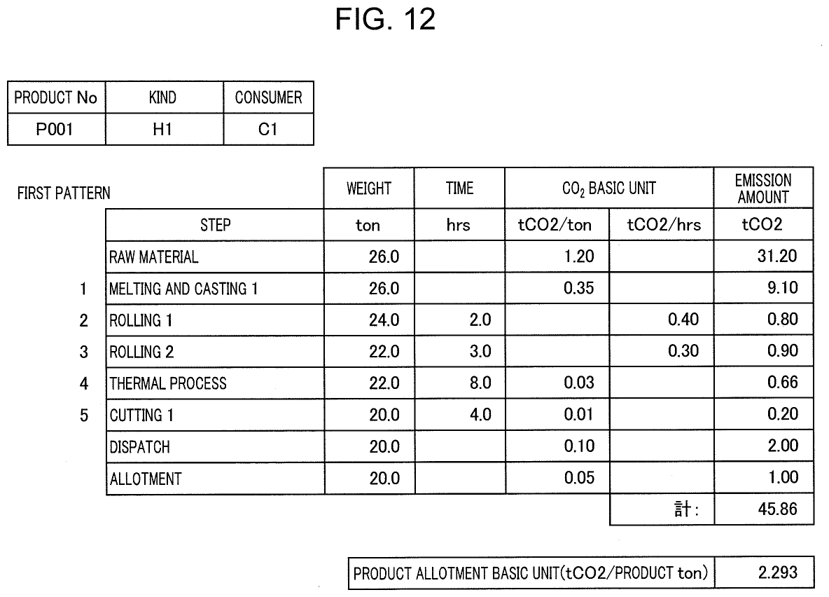

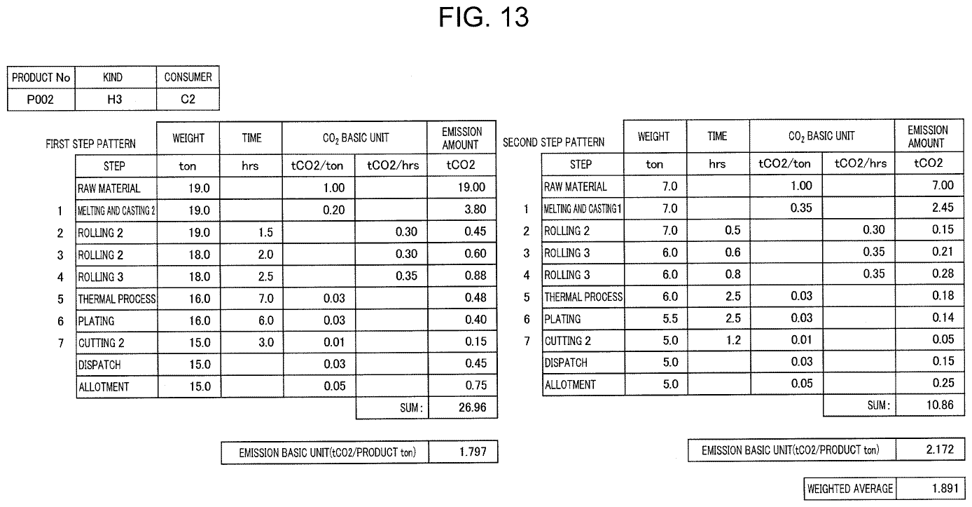

FIG. 8 illustrates an example table for explanation of a way of calculating a total step CO₂ emission amount about a step executed by a main facility and a way of calculating a total auxiliary facility CO₂ emission amount about an auxiliary facility. FIG. 9 includes illustrations of example tables for explanation of a way of calculating a CO₂ emission allotment amount about a main facility that utilizes an auxiliary facility. FIG. 10 illustrates an example table for explanation of a way of calculating the second unit CO₂ emission amount per unit about a main facility. FIG. 11 illustrates an example table for explanation of a way of calculating the fourth unit CO₂ emission amount per product unit about a remaining emission source. FIG. 12 illustrates an example output of calculation results. FIG. 13 illustrates another example output of calculation results.

Referring back to FIG. 1, the control processor 4 controls each of the parts 1 to 3, and 5 of the carbon dioxide emission amount calculation apparatus S in accordance with a function of each part, and serves as a circuit for calculating CO₂ emission amounts about a target product of a target type. The control processor 4 includes, for example, a central processing part (CPU) and its peripheral circuits. The control processor 4 enables a control part 41, a raw material emission amount calculation part 42, a main facility unit emission amount calculation part 43, a step emission amount calculation part 44, a emission allotment amount calculation part 45, a accompanying process emission amount calculation part 46, a first remaining emission amount calculation part 47, a second remaining emission amount calculation part 48, and a target product emission amount calculation part 49 to be operative in response to execution of the control processing programs.

The control part 41 is intended for control of an entirety of the carbon dioxide emission amount calculation apparatus S through control of each of the parts 1 to 3 and 5 of the carbon dioxide emission amount calculation apparatus in accordance with operability of these parts.

The raw material emission amount calculation part 42 calculates, on the basis of a given first unit CO₂ emission amount per unit about a raw material in connection with production of the target product of the target type and a total amount of the raw material used for the target product of the target type produced in a predetermined period, a total raw material CO₂ emission amount about the raw material in connection with the production of the target product of the target type.

For example, a total raw material CO₂ emission amount about a raw material is calculated for the product with the product number “P001” serving as the target product of the target type. It is noted here that the production step information table 100 has registration of a total amount of a raw material used for such a target product of a target type produced in a predetermined period in the embodiment, as described above. Hence, in this example, the raw material emission amount calculation part 42 firstly takes out or reads out, as the total amount of the raw material, an actual performance value “26 [ton]” registered in the weight field 103 in the first step from the production step information table 100a for the product with the product number “P001” as stored in the production step information storage section 51. Further, the first unit emission amount information table 500a has registration of the first unit CO₂ emission amount per unit about the raw material in association with a kind in the embodiment, as described above. Thus, the raw material emission amount calculation part 42 subsequently retrieves and selects a record having registration of the number “P001” in the product number field 201 from the product actual production performance information table 200 stored in the product actual production performance information storage section 52, and takes out a kind “H1” registered in the kind field 2021 in the selected record. Next, the raw material emission amount calculation part 42 retrieves and selects a record having registration of the kind “H1” in the kind field 501a from the first unit emission amount information table 500a stored in the unit emission amount information storage section 55, and takes out the first unit CO₂ emission amount per unit “1.2 [tCO2/ton]” about the raw material registered in the emission factor field 502a in the selected record. The raw material emission amount calculation part 42 then obtains the total raw material CO₂ emission amount “31.2 [tCO2]” about the raw material in connection with production of the target product of the target type in the predetermined period by multiplying the first unit CO₂ emission amount per unit “1.2 [tCO2/ton]” about the raw material by the total amount “26 [ton]” of the raw material used for the target product of the target type produced in the predetermined period.

The emission allotment amount calculation part 45 calculates, concerning an auxiliary facility indirectly involved in the production of the product via a specific main facility and being adoptable for a plurality of facilities, a CO₂ emission allotment amount about a certain facility that utilizes the auxiliary facility by allotting a total auxiliary facility CO₂ emission amount about the auxiliary facility in the predetermined period to each of the facilities that adopts the auxiliary facility at a predetermined proportion. In the embodiment, the emission allotment amount calculation part 45 calculates the total auxiliary facility CO₂ emission amount about the auxiliary facility in the predetermined period on the basis of a total consumed item amount of a consumed item accompanied by an operation of the auxiliary facility and accompanied by carbon dioxide emission and on the basis of a CO₂ emission amount per unit (a fifth unit CO₂ emission amount per unit) about the consumed item in the predetermined period. The predetermined proportion may be, for example, appropriately set and stored in the storage part 5 in advance, but is obtained or calculated from an actual performance value in the embodiment as described later. The predetermined proportion represents, for example, a usage proportion of a produced object produced in the auxiliary facility for each of the plurality of facilities that utilizes the auxiliary facility. Alternatively, the predetermined proportion represents, for example, a proportion of a production amount of a produced object produced in each of the facilities that utilizes the auxiliary facility.

FIG. 4 illustrates an example where each of the roll polishing facility and the boiler facility serves as the auxiliary facility. These facilities will be described in detail.

In the case of the boiler facility, the emission allotment amount calculation part 45 firstly takes out a consumption amount of a consumed item accompanied by an operation of the boiler facility from the facility-related actual performance information stored in the facility-related actual performance information storage section 53. In the example in FIG. 4, the emission allotment amount calculation part 45 firstly retrieves and selects a record having registration of a facility name “boiler” in the facility name field 302 from the facility-related actual performance information table 300 stored in the facility-related actual performance information storage section 53, and takes out a consumption amount of a consumed item registered in the consumption field 304 in the selected record. For instance, a consumption amount of electric power “2,000 [kWh]” is taken out and a consumption amount of LNG “2,000 [kg]” is taken out from the record of the name “boiler”. Subsequently, the emission allotment amount calculation part 45 takes out the fifth unit CO₂ emission amount per unit about the consumed item accompanied by the operation of the boiler facility from the unit emission amount information stored in the unit emission amount information storage section 55. In the example in FIG. 6B, the emission allotment amount calculation part 45 retrieves and selects records respectively having registration of “electric power” and “LNG” in the name field 501b from the second unit emission amount information table 500b stored in the unit emission amount information storage section 55, and takes out each fifth unit CO₂ emission amount per unit and a relevant unit about each of the electric power and the LNG registered in each of the emission factor field 503b and the unit field 502b in each selected record. The fifth unit CO₂ emission amount about electric power (electric power unit CO₂ emission amount) “0.0005 [tCO2/kWh]” is taken out. The fifth unit CO₂ emission amount about LNG (LNG unit CO₂ emission amount) “0.004 [tCO2/kg]” is taken out. Then, as illustrated in FIG. 8, the emission allotment amount calculation part 45 obtains: a total CO₂ emission amount “1 [tCO2]” about the electric power in the predetermined period by multiplying the consumption amount of electric power “2,000 [kWh]” by the electric power unit CO₂ emission amount “0.0005 [tCO2/kWh]”; a total CO₂ emission amount “8 [tCO2]” about the LNG in the predetermined period by multiplying the consumption amount of LNG “2,000 [kg]” by the LNG unit CO₂ emission amount “0.004 [tCO2/kg]”; and a total auxiliary facility CO₂ emission amount (total boiler facility CO₂ emission amount) “10 [tCO2]” about the boiler facility serving as the auxiliary facility in the predetermined period by calculating a sum of the above-obtained results. The sum of the total CO₂ emission amount “1 [tCO2]” about the electric power and the total CO₂ emission amount “8 [tCO2]” about the LNG is “9 [tCO2]”. In this regard, the total boiler facility CO₂ emission amount is defined as “10 [tCO2]” additionally incorporating the total CO₂ emission amount “1 [tCO2]” about the consumed item (not shown) for convenience of explanation below.

In the case of the boiler facility serving as the auxiliary facility, the predetermined proportion represents a usage proportion or usage rate of the produced object, produced in the auxiliary facility, in each of the facilities that utilizes the auxiliary facility. Specifically, the emission allotment amount calculation part 45 firstly takes out information about a facility that utilizes vapor produced or generated in the boiler facility from the auxiliary facility correlation information (the auxiliary facility correlation information matrix 600 in the example in FIG. 7) stored in the auxiliary facility correlation information storage section 56. In the example in FIG. 7, the information about the following facilities showing registration of the flag “1” are taken out: the main facility with the facility name “melting and casting 1”; the main facility with the facility name “melting and casting 2”; the main facility with the facility name “rolling 1”; the main facility with the facility name “rolling 2”; the main facility with the facility name “rolling 3”; the main facility with the facility name “thermal process”; the main facility with the facility name “plating”; the main facility with the facility name “cutting 2”; and the auxiliary facility with the facility name “roll polishing”. Subsequently, the emission allotment amount calculation part 45 takes out a vapor usage amount in a facility that utilizes the vapor produced in the boiler facility from the facility-related actual performance information stored in the facility-related actual performance information storage section 53. In the example in FIG. 4, the emission allotment amount calculation part 45 retrieves and selects, from the facility-related actual performance information table 300 stored in the facility-related actual performance information storage section 53, records respectively having registration of “melting and casting 1”, “melting and casting 2”, “rolling 1”,“rolling 2”, “rolling 3”, “thermal process”, “plating”, “cutting 2”, and “roll polishing” in the facility name field 302. The emission allotment amount calculation part takes out vapor usage amounts “0 [kg]”, “0 [kg]”, “400 [kg]”, “1,200 [kg]”, “800 [kg]”, “400 [kg]”, “800 [kg]”, “0 [kg]”, “0 [kg]”, and “400 [kg]” registered in the vapor field 30413 in the respective records. Then, as illustrated in FIG. 9A, the emission allotment amount calculation part 45 calculates a vapor usage proportion or vapor usage rate about the boiler facility from the taken out vapor usage amounts, and calculates, for each of the facilities that utilizes the boiler facility, a CO₂ emission allotment amount about each facility that utilizes the boiler facility by multiplying the calculated vapor usage proportion or vapor usage rate by the total boiler facility CO₂ emission amount “10 [tCO2]”. For instance, for the main facility with the facility name “rolling 1”, the vapor usage rate 0.1 is multiplied by the total boiler facility CO₂ emission amount “10 [tCO2]”, and the result “1 [tCO2]” is obtained as the CO₂ emission allotment amount. Alternatively, for instance, for the auxiliary facility with the facility name “roll polishing”, the vapor usage rate 0.1 is multiplied by the total boiler facility CO₂ emission amount “10 [tCO2]”, and the result “1 [tCO2]” is obtained as the CO₂ emission allotment amount.

In the case of the roll polishing facility, the emission allotment amount calculation part 45 firstly takes out a consumption amount of a consumed item accompanied by an operation of the roll polishing facility from the facility-related actual performance information stored in the facility-related actual performance information storage section 53. In the example in FIG. 4, the emission allotment amount calculation part 45 firstly retrieves and selects a record having registration of the facility name “roll polishing” in the facility name field 302 from the facility-related actual performance information table 300 stored in the facility-related actual performance information storage section 53, and takes out a consumption amount of a consumed item registered in the consumption field 304 in the selected record. For instance, a consumption amount of electric power “10,000 [kWh]” is taken out, a consumption amount of vapor “400 [kg]” is taken out, and a consumption amount of lubricant “5 [kL]” is taken out from the record of the name “roll polishing”. Subsequently, the emission allotment amount calculation part 45 takes out the fifth unit CO₂ emission amount per unit about the consumed item accompanied by the operation of the roll polishing facility from the unit emission amount information stored in the unit emission amount information storage section 55. As described above, the emission allotment amount calculation part 45 already obtains the total CO₂ emission amount “1 [tCO2]” about the vapor, and thus takes out the fifth unit CO₂ emission amount about “electric power” and the fifth unit CO₂ emission amount about “lubricant”. In the example in FIG. 6B, the emission allotment amount calculation part 45 retrieves and selects records respectively having registration of “electric power” and “lubricant” in the name field 501b from the second unit emission amount information table 500b stored in the unit emission amount information storage section 55, and takes out each fifth unit CO₂ emission amount per unit and a relevant unit about each of the electric power and the lubricant registered in each of the emission factor field 503b and the unit field 502b in each selected record. The fifth unit CO₂ emission amount about electric power (electric power unit CO₂ emission amount) “0.0005 [tCO2/kWh]” is taken out. The fifth unit CO₂ emission amount about lubricant (lubricant unit CO₂ emission amount) “0.8 [tCO2/kL]” is taken out. Then, as illustrated in FIG. 8, the emission allotment amount calculation part 45 obtains: a total CO₂ emission amount “5 [tCO2]” about the electric power in the predetermined period by multiplying the consumption amount of electric power “10,000 [k Wh]” by the electric power unit CO₂ emission amount “0.0005 [tCO2/kWh]”; a total CO₂ emission amount “4 [tCO2]” about the lubricant in the predetermined period by multiplying the consumption amount of lubricant “5 [kL]” by the lubricant unit CO₂ emission amount “0.8 [tCO2/kL]”; a sum of the above-obtained results; and a total auxiliary facility CO₂ emission amount (total roll polishing facility CO₂ emission amount) “10 [tCO2]” about the roll polishing facility serving as the auxiliary facility in the predetermined period by adding the total CO₂ emission amount “1 [tCO2]” about the vapor to the sum.

In the case of the roll polishing facility serving as the auxiliary facility, the predetermined proportion represents, for example, a proportion of a production amount of a produced object produced in each of the facilities that utilizes the auxiliary facility. Although FIG. 9B exemplifies each proportion of the production amount expressed with a weight, the proportion is not limited to the weight and may be appropriately set depending on, for example, a type of the produced object, and may be expressed with, for example, a volume of the produced object having been processed. Specifically, the emission allotment amount calculation part 45 firstly takes out information about a facility that utilizes the roll polishing facility from the auxiliary facility correlation information (the auxiliary facility correlation information matrix 600 in the example in FIG. 7) stored in the auxiliary facility correlation information storage section 56. In the example in FIG. 7, information about the main facility with the facility name “rolling 1”, the main facility with the facility name “rolling 2”, and the main facility with the facility name “rolling 3” each showing registration of the flag “1” are taken out. Subsequently, the emission allotment amount calculation part 45 takes out a production amount of a produced object in each facility that utilizes the roll polishing facility from the facility-related actual performance information stored in the facility-related actual performance information storage section 53. In the example in FIG. 4, the emission allotment amount calculation part 45 retrieves and selects, from the facility-related actual performance information table 300 stored in the facility-related actual performance information storage section 53, records respectively having registration of “rolling 1”, “rolling 2”, and “rolling 3” in the facility name field 302, and takes out weights “600 [ton]”, “2,000 [ton]”, and “1,400 [ton]” registered in the weight field 3031 in the records. Then, as illustrated in FIG. 9B, the emission allotment amount calculation part 45 calculates a proportion, here, a weight rate, of the production amount of the produced object from the taken out weights, and calculates, for each of the facilities that utilizes the roll polishing facility, a CO₂ emission allotment amount about each of the facilities that utilizes the roll polishing facility by multiplying the calculated proportion or weight rate of the production amount of the produced object by the total roll polishing facility CO₂ emission amount “10 [tCO2]”. For instance, for the main facility with the facility name “rolling 1, the weight rate 0.15 is multiplied by the total roll polishing facility CO₂ emission amount “10 [tCO2]”, and the result “1.5 [tCO2]” is obtained as the CO₂ emission allotment amount.

In the embodiment, the total CO₂ emission amount “1 [tCO2]” about the vapor used in the roll polishing facility among the total boiler facility CO₂ emission amount “10 [tCO2]” is allotted to a main facility on the basis of each CO₂ emission allotment amount about each facility that utilizes the roll polishing facility in a manner to be described below.

The main facility unit emission amount calculation part 43 calculates, for each of a plurality of main facilities respectively associated with the steps in the production of the target product of the target type, a second unit CO₂ emission amount per unit about each of the main facilities on the basis of a total production load amount required in the main facility associated with the step to execute the step in the production in the predetermined period and a total CO₂ emission amount about the main facility with respect to the total production load amount. In the embodiment, the main facility unit emission amount calculation part 43 calculates, for each of the main facilities respectively associated with the steps in the production of the target product of the target type, a total CO₂ emission amount about the main facility with respect to a total production load amount required in the main facility in the predetermined period in connection with the production on the basis of a total consumed item amount of a consumed item accompanied by an operation of the main facility and accompanied by carbon dioxide emission and on the basis of the fifth unit CO₂ emission amount per unit about the consumed item in the predetermined period.

For instance, the total production load amount is expressed with a total produced object amount in production in the main facility. The main facility unit emission amount calculation part 43 calculates, for each of the main facilities respectively associated with the steps in the production of the target product of the target type, the second unit CO₂ emission amount per unit about each of the main facilities, on the basis of the total produced object amount in the production in the main facility in the predetermined period and on the basis of the total CO₂ emission amount about the main facility in connection with the production for the total produced object amount. Alternatively, for example, the total production load amount is expressed with a total usage time of the main facility. The main facility unit emission amount calculation part 43 calculates, for each of the main facilities respectively associated with the steps in the production of the target product of the target type, the second unit CO₂ emission amount per unit about each of the main facilities on the basis of a total usage time required in the main facility in the predetermined period in connection with the production and the total CO₂ emission amount about the main facility in connection with the usage in the total usage time.

In the embodiment, when the main facility has an association with the auxiliary facility in the calculation of the second unit CO₂ emission amount per unit about the main facility, the main facility unit emission amount calculation part 43 calculates a new total CO₂ emission amount about the main facility with respect to the total production load amount by adding the CO₂ emission allotment amount about the facility corresponding to the main facility calculated by the emission allotment amount calculation part 45 to the total CO₂ emission amount about the main facility with respect to the total production load amount. The main facility unit emission amount calculation part then calculates the second unit CO₂ emission amount per unit about the main facility on the basis of the calculated new total CO₂ emission amount about the main facility with respect to the total production load amount.

For instance, in the case where the product with the product number “P001” serves as the target product of the target type, the main facility unit emission amount calculation part 43 firstly takes out all steps in production of the product with the product number “P001” from the production step information table 100a for the product with the product number “P001” stored in the production step information storage section 51 to obtain a plurality of steps in production of the target product of the target type. FIG. 2A exemplifies taking-out of the first step executed by the main facility with the facility name “melting and casting 1”, the second step executed by the main facility with the facility name “rolling 1”, the third step executed by the main facility with the facility name “rolling 2”, the fourth step executed by the main facility with the facility name “thermal process”, and the fifth step executed by the main facility with the facility name “cutting 12”. Subsequently, the main facility unit emission amount calculation part 43 takes out, for each of the main facilities respectively associated with the first to fifth steps, a consumption amount of a consumed item accompanied by an operation of the main facility from the facility-related actual performance information stored in the facility-related actual performance information storage section 53. In the examples in FIG. 2A and FIG. 4, the main facility unit emission amount calculation part 43 retrieves and selects records respectively having registration of the facility names “melting and casting 1”, “rolling 1”, “rolling 2”, “thermal process”, and “cutting 1” of the main facilities associated with the first to fifth steps in the facility name field 302 from the facility-related actual performance information table 300 stored in the facility-related actual performance information storage section 53, and takes out respective consumption amounts of consumed items registered in the consumption field 304 in the selected records. For instance, a consumption amount of LNG “80,000 [kg]” and a consumption amount of chemical “6,000 [thousand yen]” are taken out from the record of the name “melting and casting 1”. Subsequently, the main facility unit emission amount calculation part 43 takes out the fifth unit CO₂ emission amount per unit about the consumed item accompanied by the operation of the main facility from the unit emission amount information stored in the unit emission amount information storage section 55. In the example in FIG. 6B, the main facility unit emission amount calculation part 43 retrieves and selects records respectively having registration of “LNG” and “chemical” in the name field 501b from the second unit emission amount information table 500b stored in the unit emission amount information storage section 55, and takes out each fifth unit CO₂ emission amount per unit and a relevant unit about each of the LNG and the lubricant registered in each of the emission factor field 503b and the unit field 502b in each selected record. The fifth unit CO₂ emission amount about LNG (LNG unit CO₂ emission amount) “0.004 [tCO2/kg]” is taken out. The fifth unit CO₂ emission amount about chemical (chemical unit CO₂ emission amount) “0.01 [tCO2/thousand yen]” is taken out. Then, as illustrated in FIG. 8, the main facility unit emission amount calculation part 43 obtains: a total CO₂ emission amount “320 [ton]” about the LNG in the predetermined period by multiplying the consumption amount of LNG “80,000 [kg]” by the LNG unit CO₂ emission amount “0.004 [tCO2/kg]”; a total CO₂ emission amount “60 [tCO2]” about the chemical in the predetermined period by multiplying the consumption amount of chemical “6,000 [thousand yen]” by the chemical unit CO₂ emission amount “0.01 [tCO2/thousand yen]”; and a total CO₂ emission amount “420 [tCO2]” about the main facility with the facility name “melting and casting 1” in connection with the production for the total produced object amount in the main facility with the facility name “melting and casting 1” in the predetermined period by calculating a sum of the above-obtained results. Similarly, for the main facilities with the facility names “rolling 1”, “rolling 2”, “thermal process”, and “cutting 1”, the total CO₂ emission amounts “48 [tCO2]”, “60 [tCO2]”, “48 [tCO2]”, and “8 [tCO2]” about the main facilities in connection with the production for the total produced object amount in each main facility in the predetermined period are respectively obtained.

Here, when a certain main facility, such as the main facility with the facility name “rolling 1”, has an association with the auxiliary facility, the main facility unit emission amount calculation part 43 calculates a new total CO₂ emission amount about the main facility with respect to the total production load amount by adding the CO₂ emission allotment amount about a facility corresponding to the main facility calculated by the emission allotment amount calculation part 45 in the aforementioned manner to the total CO₂ emission amount about the main facility with respect to the total production load amount. For instance, as illustrated in FIG. 8, in the case of the main facility with the facility name “rolling 1”, the main facility unit emission amount calculation part 43 obtains: a total CO₂ emission amount “25 [tCO2]” about the electric power in the predetermined period by multiplying the consumption amount of electric power “50,000 [k Wh]” by the electric power unit CO₂ emission amount “0.0005 [tCO2/kWh]”; a total CO₂ emission amount “4 [tCO2]” about the lubricant in the predetermined period by multiplying the consumption amount of lubricant “5 [kL]” by the lubricant unit CO₂ emission amount “0.8 [tCO2/kL]”; a total CO₂ emission amount “45.5 [ton]” about the main facility with the facility name “rolling 1” with respect to the total production load amount required in the main facility with the facility name “rolling 1” in the predetermined period in connection with the production by calculating a sum of the above-obtained results. The sum of the total CO₂ emission amount “25 [tCO2]” about the electric power and the total CO₂ emission amount “4 [tCO2]” about the lubricant is “29 [tCO2]”. In this regard, the total CO₂ emission amount about the main facility with the facility name “rolling 1” is defined as “45.5 [tCO2]” additionally incorporating the total CO₂ emission amount “16.5 [tCO2]” about the consumed item (not shown) for convenience of explanation below. Moreover, as illustrated in FIG. 10, the main facility unit emission amount calculation part 43 obtains a new total CO₂ emission amount “48 [tCO2]” about the main facility with the facility name “rolling 1” by adding: the CO₂ emission allotment amount “1 [tCO2]” by the boiler facility for the main facility with the facility name “rolling 1”; and the CO₂ emission allotment amount “1.5 [tCO2”] by the roll polishing facility for the main facility with the facility name “rolling 1” to the total CO₂ emission amount “45.5 [tCO2] about the main facility with the facility name “rolling 1”, each of the CO₂ emission allotment amounts being calculated by the emission allotment amount calculation part 45 about a facility corresponding to the main facility.

Subsequently, the main facility unit emission amount calculation part 43 takes out, for each of the main facilities respectively associated with the first to fifth steps, a total production load amount required in the main facility in the predetermined period in connection with the production from the facility-related actual performance information stored in the facility-related actual performance information storage section 53. In the examples in FIG. 2A and FIG. 4, the main facility unit emission amount calculation part 43 retrieves and selects records respectively having registration of the facility names “melting and casting 1”, “rolling 1”, “rolling 2”, “thermal process”, and “cutting 1” of the main facilities associated with the first to fifth steps in the facility name field 302 from the facility-related actual performance information table 300 stored in the facility-related actual performance information storage section 53, and takes out a total production load amount registered in the production load field 303 in each of the selected records. For instance, a total produced project amount “1,200 [ton]” is taken out from the record of the facility name “melting and casting 1”. Alternatively, for instance, a total produced object amount “600 [ton]” is taken out and a total usage time “120 [hrs]” is taken out from the record of the facility name “rolling 1”.

Then, the main facility unit emission amount calculation part 43 calculates, for each of the main facilities respectively associated with first to fifth steps, the second unit CO₂ emission amount per unit about the main facility on the basis of the total production load amount required in the main facility in the predetermined period in connection with the production as obtained in the aforementioned manner and on the basis of the total CO₂ emission amount about the main facility with respect to the total production load amount as obtained in the aforementioned manner. For instance, as illustrated in FIG. 10, for the main facility with the facility name “melting and casting 1”, the main facility unit emission amount calculation part 43 obtains the second unit CO₂ emission amount (melting and casting 1-unit CO₂ emission amount) per unit “0.35[tCO2 /ton]” about the main facility with the facility name “melting and casting 1” by dividing the total CO₂ emission amount “420 [tCO2”] about the main facility with the facility name “melting and casting 1” with respect to the total production load amount by the total produced object amount “1,200 [ton]” as a total production load amount required in the main facility with the facility name “melting and casting 1” in the predetermined period in connection with the production (420/1,200=0.35). Besides, for instance, the main facility unit emission amount calculation part 43 obtains, for the main facility with the facility name “rolling 1”, the second unit CO₂ emission amounts (rolling 1-unit CO₂ emission amounts) per unit “0.08[tCO2 /ton]” and “0.4[tCO2 /hrs]” about the main facility with the facility name “rolling 1” by dividing the total CO₂ emission amount “48 [tCO2]” about the main facility with the facility name “rolling 1” with respect to the total production loading amount respectively by the total produced object amount “600 [ton]” and the total usage time “120 [hrs]” each serving as the total production load amount required in the main facility with the facility name “rolling 1” in the predetermined period in connection with the production (48/600=0.08, 48/120=0.4). Similarly, for the main facilities with the facility names “rolling 2”, “thermal process”, and “cutting 1”, the second unit CO₂ emission amounts “0.03 [tCO2/ton]” and “0.3 [tCO2/hrs]”, “0.03 [tCO2/ton]”, and “0.01 [tCO2/ton]” are obtained.

Both the total produced object amount “600 [ton]” and the total usage time “120 [hrs]” may be taken out to each serve as the total production load amount about the main facility with the facility name “rolling 1” as described above, and the second unit CO₂ emission amount may be calculated for each of the units [tCO2/ton] and [tCO2/hrs]. Alternatively, only one of the total amount and the total time usage may be taken out to be adopted for the second unit CO₂ emission amount per unit about the main facility with the facility name “rolling 1”, and the second unit CO₂ emission amount per unit for the taken out unit may be calculated. For instance, only the total usage time “120 [hrs]” may be taken out as the total production load amount about the main facility with the facility name “rolling 1”, and only the melting and casting 1-unit CO₂ emission amount “0.4 [tCO2/hrs]” about the main facility with the facility name “rolling 1” may be obtained. A user or operator may appropriately preset as to which of the second unit CO₂ emission amounts at the units [tCO2/ton] and [tCO2/hrs] is adopted. For instance, the setting is made through an input from the input part 1 into the carbon dioxide emission amount calculation apparatus S, and the main facility unit emission amount calculation part 43 calculates the second unit CO₂ emission amount at the set unit.

FIG. 8 to FIG. 10 show data of facilities in addition to the data of the main facilities with the facility names “melting and casting 1”, “rolling 1”, “rolling 2”, “thermal process”, and “cutting 1”, and the auxiliary facilities with the facility names “roll polishing” and the “boiler”.

Referring back to FIG. 1, the step emission amount calculation part 44 calculates, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated by the main facility unit emission amount calculation part 43 and the total production load amount required in the main facility in the predetermined period in connection with the production of the target product of the target type. In the case where the total production load amount is expressed with the total produced object amount in the main facility in connection with the production, the step emission amount calculation part 44 calculates, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated by the main facility unit emission amount calculation part and the total produced object amount in the main facility in the predetermined period in connection with the production of the target product of the target type. In the case where the total production load amount is expressed with the total usage time about the main facility, the step emission amount calculation part 44 calculates, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated by the main facility unit emission amount calculation part and the total usage time required in the main facility in the predetermined period in connection with the production of the target product of the target type.

For instance, in the case where the product with the product number “P001” serves as the target product of the target type, the production step information table 100 has registration of the total production load amount required in the main facility in the predetermined period in connection with the production of the target product of the target type in the embodiment, as described above. In this regard, the step emission amount calculation part 44 firstly takes out, for each of the main facilities respectively associated with the first to fifth steps, the total production load amount required in the main facility in the predetermined period in connection with the production of the target product of the target type from the production step information table 100a for the product with the product number “P001” stored in the production step information storage section 51. In the example, the total production load amounts “26 [ton]”, “2 [hrs], “3 [hrs]”, “22 [ton]”, and “20 [ton]” are obtained respectively for the main facilities with the facility names “melting and casting 1”, “rolling 1”, “rolling 2”, “thermal process”, and “cutting 1”.

Subsequently, the step emission amount calculation part 44 calculates, for each of the main facilities respectively associated with the first to fifth steps, a step CO₂ emission amount about a step executed by the main facility in connection with production of the target product of the target type by multiplying the second unit CO₂ emission amount per unit about the main facility calculated by the main facility unit emission amount calculation part 43 in the aforementioned manner by the total production load amount required in the main facility in the predetermined period in connection with the production of the target product of the taken target type. In this example, for the product with the product number “P001”, the step CO₂ emission amount “9.1 [tCO 2]” (=0.35 [tCO2/ton]×26 [ton]) about the step executed by the main facility with the facility name “melting and casting 1” is obtained. The step CO₂ emission amount “0.8 [tCO2]” (=0.4 [tCO2/hrs]×2 [hrs]) about the step executed by the main facility with the facility name “rolling 1” is obtained. The step CO₂ emission amount “0.9 [tCO2]” (=0.3[tCO2 /hrs]×3 [hrs]) about the step executed by the main facility with the facility name “rolling 2” is obtained. The step CO₂ emission amount “0.66 [tCO2]” (=0.03[tCO2 /ton] ×22 [ton]) about the step executed by the main facility with the facility name “thermal process” is obtained. The step CO₂ emission amount “0.2 [tCO2]” (=0.01[tCO2 /ton]×20 [ton]) about the step executed by the main facility with the facility name “cutting 1” is obtained.

The accompanying process emission amount calculation part 46 calculates, on the basis of a given third unit CO₂ emission amount per unit about a predetermined process executed to the product after the production of the product to delivery of the product to a delivery destination and a total amount of the target product of the target type produced in the plant in the predetermined period, a total accompanying process CO₂ emission amount about the predetermined process to the target product of the target type. The accompanying process emission amount calculation part 46 calculates the CO₂ emission amount about the predetermined process to the target product of the target type in the predetermined period.

As described above, examples of the predetermined process include an inspection process, a wrapping process, and a transport process. Here, as an example, the transport process in the case of the product with the product number “P001” serving as the target product of the target type will be described in more detail. The third unit emission amount information table 500c stored in the unit emission amount information storage section 55 has registration of a third unit CO₂ emission amount (transport process unit CO₂ emission amount) per unit about the transport process in association with a consumer in the embodiment. Hence, the accompanying process emission amount calculation part 46 retrieves and selects records having registration of the number “P001” in the product number field 201 from the product actual production performance information table 200 stored in the product actual production performance information storage section 52, and takes out a consumer “C1” and a production amount “20 [ton]” respectively registered in a consumer field 2022 and the production amount field 203 in the selected records. Next, the accompanying process emission amount calculation part 46 retrieves and selects a record having registration of “C1” in the consumer field 501c from the third unit emission amount information table 500c stored in the unit emission amount information storage section 55, and takes out the transport process unit CO₂ emission amount “0.1 [tCO2/ton]” registered in the emission factor field 502c in the selected record. The accompanying process emission amount calculation part 46 defines the taken out production amount “20 [ton]” as a transport amount, and obtains the total accompanying process CO₂ emission amount “2 [tCO2]” (=0.1[tCO2 /ton]×20 [ton]) about the transport process to the product with the product number “P001” by multiplying the taken out transport process unit CO₂ emission amount “0.1 [tCO2/ton]” by the transport amount or production amount “20 [ton]”.

The first remaining emission amount calculation part 47 calculates a fourth unit CO₂ emission amount per product unit about the remaining emission source on the basis of a total remaining CO₂ emission amount about the remaining emission source in the predetermined period and a total amount of the product produced in the plant in the predetermined period. The first remaining emission amount calculation part 47 calculates a total remaining CO₂ emission amount about the remaining emission source on the basis of a total consumed item amount of a consumed item accompanied by an operation of the remaining emission source and accompanied by carbon dioxide emission and on the basis of a fifth unit CO₂ emission amount per unit about the consumed item in the predetermined period.

The second remaining emission amount calculation part 48 calculates a total second remaining CO₂ emission amount about the remaining emission source in connection with the production of the target product of the target type on the basis of the fourth unit CO₂ emission amount per product unit about the remaining emission source calculated by the first remaining emission amount calculation part 47 and a total amount of the target product of the target type produced in the predetermined period.

As described above, examples of the remaining emission source include an in-plant conveyor, a device in a common facility accompanied by CO₂ emission, and an industrial waste processing facility. The remaining emission source is not directly related to a specific product. Hence, a CO₂ emission amount about the remaining emission source is not directly allottable to such a specific product. In this regard, in the embodiment, the CO₂ emission amount about the remaining emission source is related to an entirety of the product produced in the plant in the predetermined period. The first remaining emission amount calculation part 47 calculates a CO₂ emission amount per product unit, i.e., the fourth unit CO₂ emission amount per product unit. The second remaining emission amount calculation part 48 calculates a CO₂ emission amount to be allotted to the target product of the target type at the unit CO₂ emission amount per product unit.

For instance, in the embodiment, the remaining emission source-related actual performance information storage section 54 stores a consumption amount of a consumed object at the remaining emission source. The first remaining emission amount calculation part 47 thus takes out, for each of all remaining emission sources, a consumption amount of a consumed item from the remaining emission source-related actual performance information stored in the remaining emission source-related actual performance information storage section 54. In FIG. 5, examples of the remaining emission source include a remaining emission source with a name “in-plant conveyance”, a remaining emission source with a name “common facility”, and a remaining emission source with a name “industrial waste process”. The first remaining emission amount calculation part 47 takes out each consumption amount of each consumed item registered in the consumption field 402 in each record from the remaining emission source-related actual performance information table 400 stored in the remaining emission source-related actual performance information storage section 54. As illustrated in FIG. 11, the consumption amount of electric power “3,000 [k Wh]” and the consumption amount of light oil “10 [kL]” are taken out for the remaining emission source with the name “in-plant conveyance”, the consumption amount of electric power “1,000 [kWh]” is taken out for the remaining emission source with the name “common facility”, and the consumption amount of light oil “1 [kL]” and the consumption amount of expenses “5,000 [thousand yen] are taken out for the remaining emission source with the name “industrial waste process”. Subsequently, the first remaining emission amount calculation part 47 takes out the fifth unit CO₂ emission amount per unit about the consumed item accompanied by an operation of each remaining emission source from the second unit emission amount information stored in the unit emission amount information storage section 55. In the example in FIG. 6B, the electric power unit CO₂ emission amount “0.005 [tCO2/kWh]” is taken out, the fifth unit CO₂ emission amount about the light oil (light oil unit CO₂ emission amount) “3 [tCO2/kg]” is taken out, and the fifth unit CO₂ emission amount about the expenses (industrial waste process expenses unit CO₂ emission amount) “0.008 [tCO2/thousand yen]” required in the industrial waste process is taken out. Then, as illustrated in FIG. 11, the main facility unit emission amount calculation part 43 obtains: for the remaining emission source with the name “in-plant conveyance”, the CO₂ emission amount “1.5 [CO2]” by multiplying the electric power unit CO₂ emission amount “0.0005 [tCO2/kWh]” by the consumption amount of electric power “3,000 [kWh]”, and the CO₂ emission amount “30 [tCO2]” by multiplying the light oil unit CO₂ emission amount “3 [tCO2/kL]” by the consumption amount of light oil “10 [kL]”; for the remaining emission source with the name “common facility”, the CO₂ emission amount “0.5 [tCO2]” by multiplying the electric power unit CO₂ emission amount “0.0005 [tCO2/kWh]” by the electric power consumption amount “1,000 [kWh]”; for the remaining emission source with the name “industrial waste process”, the CO₂ emission amount “3 [tCO2]” by multiplying the light oil unit CO₂ emission amount “3 [tCO2/kL]” by the consumption amount of light oil “1 [kL]”, and the CO₂ emission amount “40 [tCO2]” by multiplying the industrial waste process expenses unit CO₂ emission amount “0.008 [tCO2/thousand yen]” by the consumption amount of expenses “5,000 [thousand yen]” required in the industrial waste process; and the total remaining CO₂ emission amount “75 (=1.5+30+0.5+3+40) [tCO2]” about the remaining emission sources in the predetermined period by calculating a sum of the above-obtained results. The main facility unit emission amount calculation part 43 further calculates a total amount of the product produced in the plant in the predetermined period from the product actual production performance information stored in the product actual production performance information storage section 52. In the example in FIG. 3, the main facility unit emission amount calculation part 43 calculates the total amount of the product, i.e., obtains “1,500 [ton]” as described above in this example, produced in the plant in the predetermined period by calculating a total production amount registered in each record in the production amount field 203 in the product actual production performance information table 200 stored in the product actual production performance information storage section 52. The main facility unit emission amount calculation part 43 obtains the fourth unit CO₂ emission amount “0.05 [tCO2/ton]” per product unit about the remaining emission source by dividing the total remaining CO₂ emission amount “75 [tCO2]” about the remaining emission sources in the predetermined period as obtained in the aforementioned manner by the total amount “1,500 [ton]” of the product produced in the plant in the predetermined period as obtained in the aforementioned manner.

For example, a CO₂ emission amount to be allotted to the product with the product number “P001” serving as the target product of the target type is calculated with the obtained fourth unit CO₂ emission amount “0.05 [tCO2/ton]” per product unit. In this example, the second remaining emission amount calculation part 48 retrieves and selects a record having registration of the number “P001” in the product number field 201 from the product actual production performance information table 200 stored in the product actual production performance information storage section 52, and takes out the production amount “20 [ton]” registered in the production amount field 203 in the selected record. The second remaining emission amount calculation part then obtains the total second remaining CO₂ emission amount “1 [tCO2]” about the remaining emission source in connection with the product with the product number “P001” by multiplying the fourth unit CO₂ emission amount “0.05 [tCO2/ton”] per product unit about the remaining emission source obtained by the first remaining emission amount calculation part 47 by the taken out total amount or production amount “20 [ton]” of the product with the product number “P001” in the predetermined period.

The target product emission amount calculation part 49 calculates, as a total target product CO₂ emission amount about the target product of the target type, a sum of: a total of respective step CO₂ emission amounts calculated by the step emission amount calculation part 44 about the steps executed by the main facilities for each of the main facilities respectively associated with the steps in the production of the target product of the target type; and the total raw material CO₂ emission amount about the raw material calculated by the raw material emission amount calculation part 42 in connection with the production of the target product of the target type. For instance, in the case where the product with the product number “P001” serves as the target product of the target type, the target product emission amount calculation part 49 obtains, as the total target product CO₂ emission amount about the product with the product number “P001”, the sum “42.86 [tCO2”] of: the total “11.66 [tCO2]” of the step CO₂ emission amount “9.1 [tCO2]” about the main facility with the facility name “melting and casting 1”, the step CO₂ emission amount “0.8 [tCO2]” about the main facility with the facility name “rolling 1”, the step CO₂ emission amount “0.9 [tCO2]” about the main facility with the facility name “rolling 2”, the step CO₂ emission amount “0.66 [tCO2]” about the main facility with the facility name “thermal process”, and the step CO₂ emission amount “0.2 [tCO2]” about the main facility with the facility name “cutting 1”, as obtained in the aforementioned manner; and the total raw material CO₂ emission amount “31.2 [tCO2]” about the raw material for the product with the product number “P001”, as obtained in the aforementioned manner.

In the embodiment, the target product emission amount calculation part 49 further calculates a new total target product CO₂ emission amount about the target product of the target type by adding the total accompanying process CO₂ emission amount about the predetermined process to the target product of the target type calculated by the accompanying process emission amount calculation part 46 and the total second remaining CO₂ emission amount about the remaining emission source in connection with the target product of the target type calculated by the second remaining emission amount calculation part 48 to the calculated total target product CO₂ emission amount about the target product of the target type. Specifically, in the embodiment, the target product emission amount calculation part 49 calculates, as a total target product CO₂ emission amount about the target product of the target type, a sum of: a total of respective step CO₂ emission amounts calculated by the step emission amount calculation part 44 about the steps executed by the main facilities for each of the main facilities respectively associated with the steps in the production of the target product of the target type; the total raw material CO₂ emission amount about the raw material calculated by the raw material emission amount calculation part 42 in connection with the production of the target product of the target type; the total accompanying process CO₂ emission amount about the predetermined process to the target product of the target type calculated by the accompanying process emission amount calculation part 46; and the total second remaining CO₂ emission amount about the remaining emission source in connection with the target product of the target type calculated by the second remaining emission amount calculation part 48. For instance, in the case where the product with the product number “P001” serves as the target product of the target type, the new total target product CO₂ emission amount “45.86 [tCO2]” about the product with the product number “P001” is obtained by adding the total accompanying process CO₂ emission amount “2 [tCO2]” about the transport process to the product with the product number “P001” as obtained by the accompanying process emission amount calculation part 46 in the aforementioned manner and the total second remaining CO₂ amount “1 [tCO2]” about the remaining emission source in connection with the product with the product number “P001” as obtained by the second remaining emission amount calculation part 48 in the aforementioned manner to the total target product CO₂ emission amount “42.86 [tCO2]” about the product with the product number “P001” as obtained in the aforementioned manner.

Although both the total accompanying process CO₂ emission amount about the predetermined process to the target product of the target type calculated by the accompanying process emission amount calculation part 46 and the total second remaining CO₂ emission amount about the remaining emission source in connection with the target product of the target type as calculated by the second remaining emission amount calculation part 48 are added in the calculation described above, one of the CO₂ emission amounts may be added.

In the embodiment, the target product emission amount calculation part 49 further calculates a sixth unit CO₂ emission amount per product unit about the target product of the target type on the basis of the obtained total target product CO₂ emission amount about the target product of the target type and the total amount of the target product of the target type produced in the plant in the predetermined period. For instance, in the case where the product with the product number “P001” serves as the target product of the target type, the sixth unit CO₂ emission amount “2.293 [tCO2/product ton] per product unit about the product with the product number “P001” is obtained by dividing the obtained target product CO₂ emission amount “45.86 [tCO2]” about the product number “P001” by the production amount “20 [ton]” of the product with the product number “P001”.

The control part 41 then outputs respective calculation results of the calculation parts 42 to 49 from the output part 2. For example, in a case where the output part 2 is in the form of a display device, the display device displays the calculation results of the calculation parts 42 to 49 on a screen of the device. Alternatively, for example, in a case where the output part 2 is in the form of a printing device, the printing device prints out the calculation results of the calculation parts 42 to 49 as a report. Each of FIG. 12 and FIG. 13 shows a relevant example. FIG. 12 illustrates a screen image or a report for the product number “P001”. FIG. 13 illustrates a screen image or a report for the product number “P002”. Each of the drawings shows: each product number of each product; a type (a kind and a consumer) of the product; steps; a total raw material amount (actual performance value) in a predetermined period; a production load (a weight and a usage time) (actual performance value) in each step (each main facility) in the predetermined period; the second unit CO₂ emission amount per unit about each main facility; a step CO₂ emission amount about each step executed by each main facility in the predetermined period; a total accompanying process CO₂ emission amount about a transport process; the fourth unit CO₂ emission amount per product unit about a remaining emission source; the total second remaining CO₂ emission amount about the remaining emission source; a total target product CO₂ emission amount; and the sixth unit CO₂ emission amount per product unit.

The control part 41 may output the calculation results of the calculation parts 42 to 49 to an external device via the IF part 3.

The input part 1, the output part 2, the IF part 3, the control processor 4, and the storage part 5 in the carbon dioxide emission amount calculation apparatus S serving as one example of the carbon dioxide emission amount calculation system are configurable by, for example, a desktop computer, a lap-top computer, or a tablet computer. Of course, the carbon dioxide emission amount calculation system may be configured by a plurality of computers communicably connected to each other as described above.

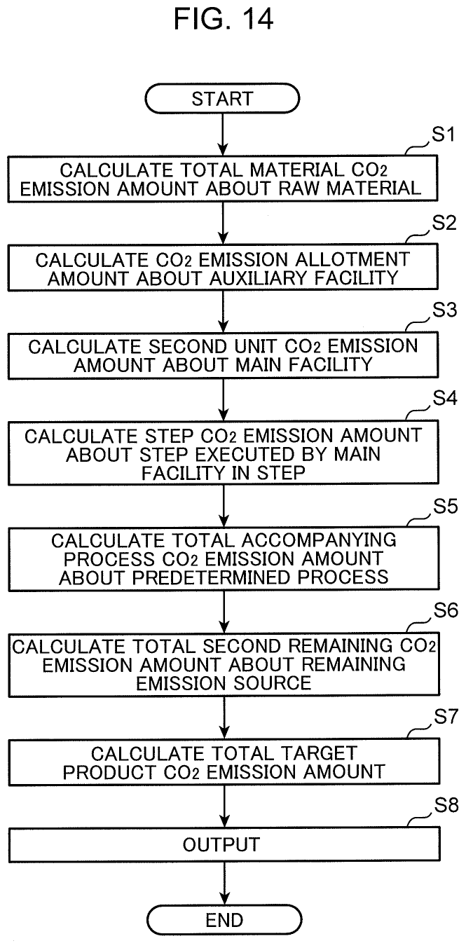

Next, an operation in the embodiment will be described. FIG. 14 is a flowchart showing an operation of a carbon dioxide emission amount calculation system (the carbon dioxide emission amount calculation apparatus serving as one example of the system).

The carbon dioxide emission amount calculation apparatus S having this configuration is powered on to execute initialization of each part which is necessary and start an operation of each part. The control processor 4 enables the control part 41, the raw material emission amount calculation part 42, the main facility unit emission amount calculation part 43, the step emission amount calculation part 44, the emission allotment amount calculation part 45, the accompanying process emission amount calculation part 46, the first remaining emission amount calculation part 47, the second remaining emission amount calculation part 48, and the target product emission amount calculation part 49 to be operative in response to execution of the control processing programs.

The carbon dioxide emission amount calculation apparatus S starts to calculate CO₂ emission amounts about a target product of one target type in response to an input of the target product of the target type by a user or operator. Here, CO₂ emission amounts about products of all the various types produced in a relevant period may be calculated. In such a case, steps SI to S8 for a target product of one target type to be described below is executed for each product of each target type.

In FIG. 14, the carbon dioxide emission amount calculation apparatus S firstly makes the raw material emission amount calculation part 42 of the control processor 4 calculate a total raw material CO₂ emission amount about a raw material in connection with production of the target product of the target type (S1).

Subsequently, the carbon dioxide emission amount calculation apparatus S makes the emission allotment amount calculation part 45 of the control processor 4 calculate, for each of a plurality of facilities that utilizes an auxiliary facility, a CO₂ emission allotment amount about the facility that utilizes the auxiliary facility (S2).

Then, the carbon dioxide emission amount calculation apparatus S makes the main facility unit emission amount calculation part 43 of the control processor 4 calculate, for each of the main facilities respectively associated with a plurality of steps in the production of the target product of the target type, a second unit CO₂ emission amount per unit about the main facility (S3).

The carbon dioxide emission amount calculation apparatus S further makes the step emission amount calculation part 44 of the control processor 4 calculate, for each of the main facilities respectively associated with the steps in the production of the target product of the target type, a step CO₂ emission amount about a step executed by the main facility in connection with the production of the target product of the target type (S4).

Subsequently, the carbon dioxide emission amount calculation apparatus S makes the accompanying process emission amount calculation part 46 of the control processor 4 calculate a total accompanying process CO₂ emission amount about a predetermined process to the target product of the target type (S5).

Thereafter, the carbon dioxide emission amount calculation apparatus S makes the first and second remaining emission amount calculation parts 47 and 48 of the control processor 4 respectively calculate a fourth unit CO₂ emission amount per product unit about a remaining emission source and a total second remaining CO₂ emission amount about the remaining emission source in connection with the target product of the target type (S6).

The carbon dioxide emission amount calculation apparatus S then makes the target product emission amount calculation part 49 of the control processor 4 calculate a total target product CO₂ emission amount about the target product of the target type (S7).

Finally, the carbon dioxide emission amount calculation apparatus S makes the control part 41 of the control processor 4 control the output part 2 output calculation results of the calculation parts 42 to 49 therefrom (step S8), and finishes the operation.

As described heretofore, the carbon dioxide emission amount calculation system (the carbon dioxide emission amount calculation apparatus serving as one example of the system) S and the carbon dioxide emission amount calculation method for the system enable calculation of a carbon dioxide emission amount per target product of each target type even without the above-described association.

A factory or plant manages total data including actual performance values in a predetermined period in many cases. First, the carbon dioxide emission amount calculation system (the carbon dioxide emission amount calculation apparatus) S and the carbon dioxide emission amount calculation method include calculating, for each of a plurality of main facilities respectively associated with a plurality of steps in production of a target product of a target type, a second unit CO₂ emission amount per unit for each of the main facilities on the basis of a total production load amount required in the main facility associated with the step to execute the step in the production in the predetermined period and a total CO₂ emission amount about the main facility with respect to the total production load amount. In this manner, the second unit CO₂ emission amount per unit about each main facility is recognizable. Hence, the carbon dioxide emission amount calculation system (the carbon dioxide emission amount calculation apparatus) S and the carbon dioxide emission amount calculation method enable calculation of, for each of the main facilities respectively associated with the steps in the production of the target product of the target type, a total step CO₂ emission amount about the step executed by the main facility in connection with the production of the target product of the target type on the basis of the second unit CO₂ emission amount per unit about the main facility calculated in the aforementioned manner and the total production load amount required in the main facility in the predetermined period in connection with the production of the target product of the target type. Accordingly, the carbon dioxide emission amount calculation system (the carbon dioxide emission amount calculation apparatus) S and the carbon dioxide emission amount calculation method enable calculation of the carbon dioxide emission amount per target product of each target type even without the aforementioned association.

In particular, a typical plant (various-kind product production plant) configured to produce a certain product of a plurality of types to be produced through a plurality of steps with a plurality of types has a plurality of different steps depending on a type of product, and thus may fail to calculate the carbon dioxide emission amount per product only through equal allotment of the total carbon dioxide emission amount about the entire plant to each product. By contrast, the carbon dioxide emission amount calculation system (the carbon dioxide emission amount calculation apparatus) S and the carbon dioxide emission amount calculation method include calculating, for each of the main facilities respectively associated with the steps, the second unit CO₂ emission amount (i.e., a unit CO₂ emission amount for each step) about the main facility, and hence enable calculation of the carbon dioxide emission amount per product even in such a various-kind product production plant.

An auxiliary facility is adoptable for a plurality of facilities. Thus, the total auxiliary facility CO₂ emission amount about the auxiliary facility in the predetermined period cannot be directly allotted to the target product of the target type. The carbon dioxide emission amount calculation system (the carbon dioxide emission amount calculation apparatus) S and the carbon dioxide emission amount calculation method include allotting the total auxiliary facility CO₂ emission amount about the auxiliary facility in the predetermined period to each of the facilities that utilizes the auxiliary facility at a predetermined proportion, and thus enable the allotment to the target product of the target type.

The carbon dioxide emission amount calculation system (the carbon dioxide emission amount calculation apparatus) S and the carbon dioxide emission amount calculation method enable incorporation of the total accompanying process CO₂ emission amount about a predetermined process executed to the product after the production of the product to delivery of the product to a delivery destination into carbon emission amounts per target product of each target type.

The carbon dioxide emission amount calculation system (the carbon dioxide emission amount calculation apparatus) S and the carbon dioxide emission amount calculation method enable incorporation of the total remaining CO₂ emission amount about the remaining emission source into the carbon dioxide emission amount per target product of each target type.

The carbon dioxide emission amount calculation system (the carbon dioxide emission amount calculation apparatus) S and the carbon dioxide emission amount calculation method include calculating a sum of: a total of respective step CO₂ emission amounts about the steps executed by the main facilities calculated for each of the main facilities respectively associated with the steps in the production of the target product of the target type; and the calculated total raw material CO₂ emission amount about the raw material in connection with the production of the target product of the target type, and thus enable calculation of the total target product CO₂ emission amount about the target product of the target type. As described above, in the case where a certain main facility utilizes an auxiliary facility, a relevant step CO₂ emission amount about a step executed by the main facility incorporates a CO₂ emission amount about the auxiliary facility. In this case, the carbon dioxide emission amount calculation system (the carbon dioxide emission amount calculation apparatus) S and the carbon dioxide emission amount calculation method enable calculation of the total target product CO₂ emission amount about the target product of the target type by incorporating the CO₂ emission amount about the auxiliary facility. In the embodiment, the carbon dioxide emission amount calculation system (the carbon dioxide emission amount calculation apparatus) S and the second carbon dioxide emission amount calculation method include calculating a new total target product CO₂ emission amount about the target product of the target type by adding the total accompanying process CO₂ emission amount about the predetermined process to the target product of the target type calculated by the accompanying process emission amount calculation part 46 and the total second remaining CO₂ emission amount about the remaining emission source in connection with the target product of the target type calculated by the second remaining emission amount calculation part 48 to the calculated total target product CO₂ emission amount about the target product of the target type. The carbon dioxide emission amount calculation system (the carbon dioxide emission amount calculation apparatus) S and the carbon dioxide emission amount calculation method enable calculation of the total target product CO₂ emission amount about the target product of the target type by incorporating the CO₂ emission amount about the predetermined process executed to the product after the production of the product to delivery of the product to a delivery destination and the CO₂ emission amount about the remaining emission source.

Meanwhile, in producing such a product made of a raw material through a plurality of steps, an object to be removed as an unavailable product without dispatch due to any defect after a finish of a certain step of the steps may be produced. The object will be referred to as a “rejected lot object” hereafter. The rejected lot object is also accompanied by carbon dioxide emission, and thus, a CO₂ emission amount about the rejected lot object is required to be incorporated. In the embodiment, CO₂ emission amounts about a target product of a target type is calculated. In this respect, the rejected lot object having failed to be a product cannot directly serve as the target product of the target type. This makes it impossible to incorporate a CO₂ emission amount about the rejected lot object. In the embodiment, any one of first to fourth addition ways to be described later may be employed to add the CO₂ emission amount about the rejected lot object to the CO₂ emission amounts about the target product of the target type so that the CO₂ emission amount about the rejected lot object is incorporated. Hereinafter, modified embodiments concerning these ways will be described in more detail. In each of the first to fourth addition ways, the control part 41, the raw material emission amount calculation part 42, the main facility unit emission amount calculation part 43, the step emission amount calculation part 44, the emission allotment amount calculation part 45, the accompanying process emission amount calculation part 46, the first remaining emission amount calculation part 47, the second remaining emission amount calculation part 48, and the target product emission amount calculation part 49 in the control processor 4 are operative as described in the embodiment.

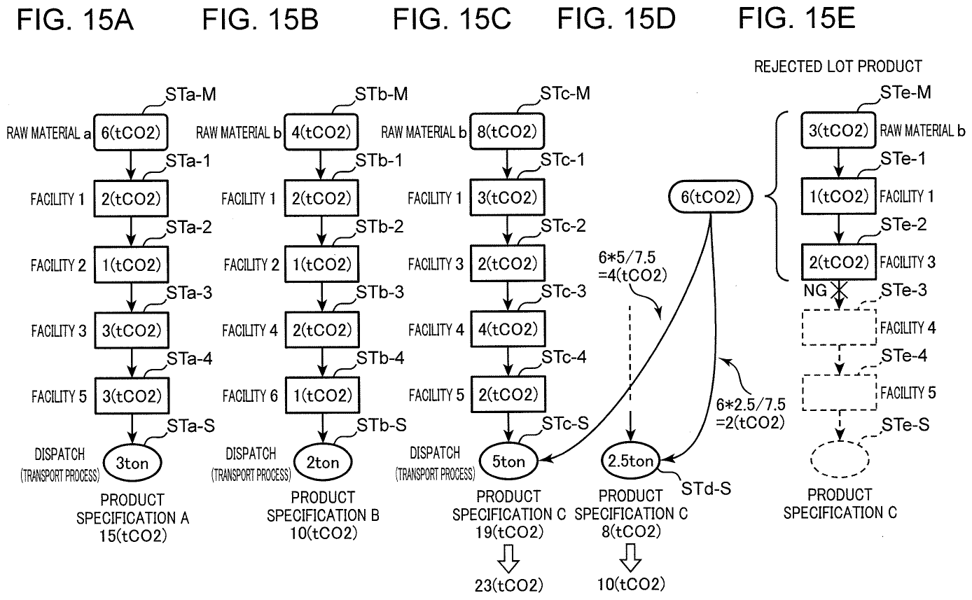

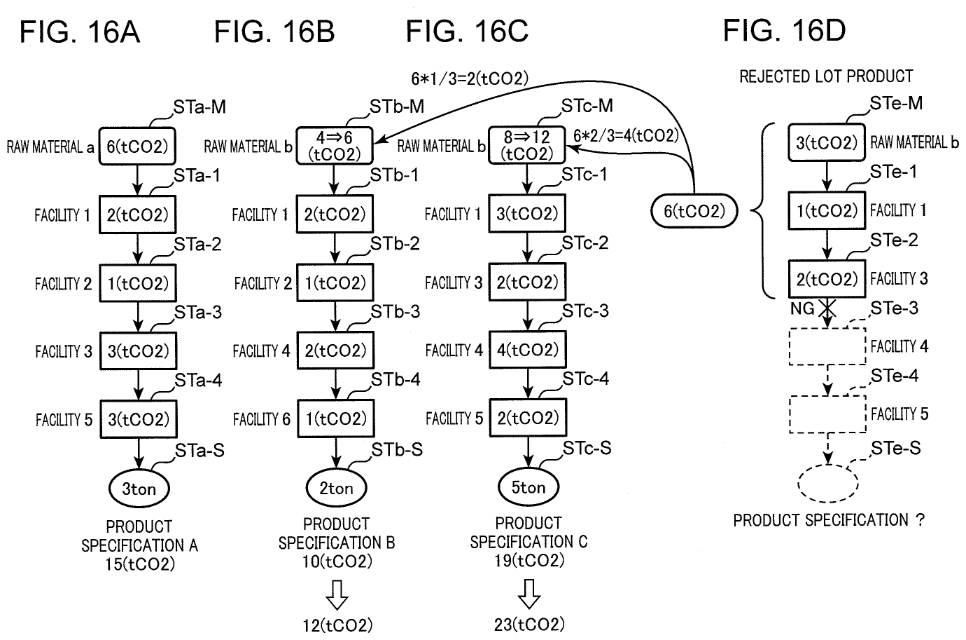

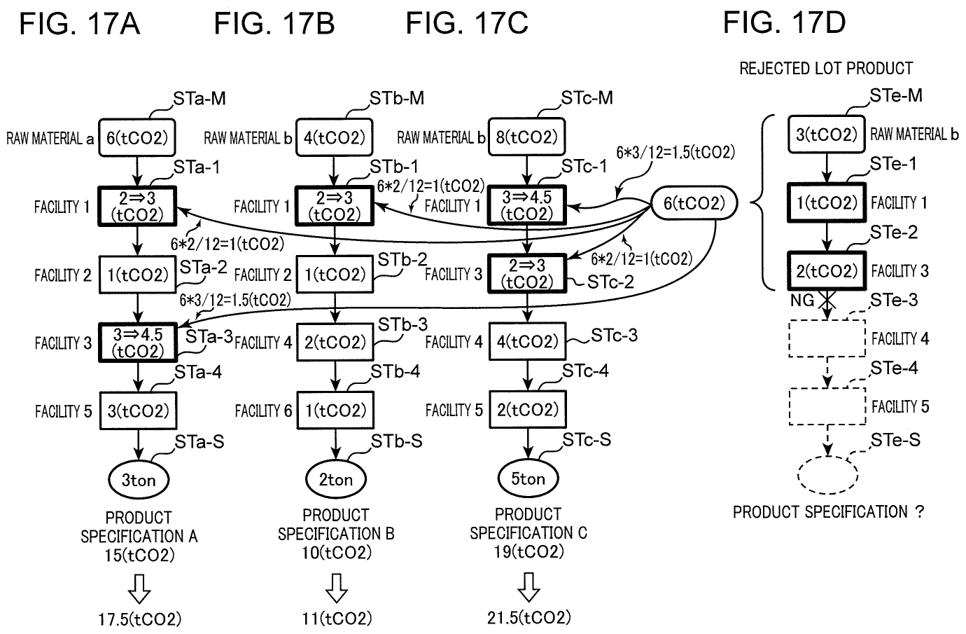

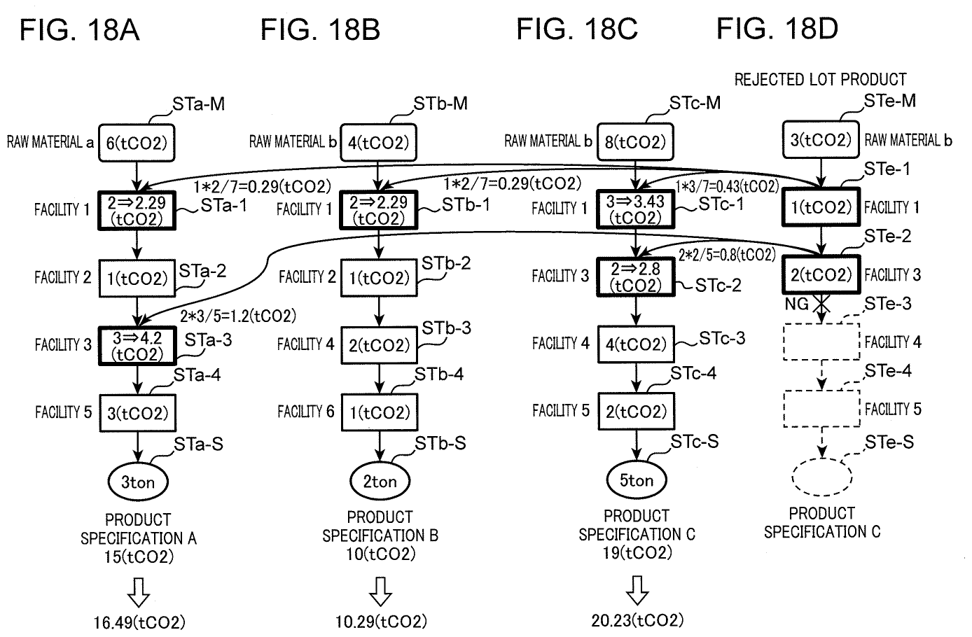

FIG. 15 includes illustrations for explanation of the first addition way in a modified embodiment. FIG. 16 includes illustrations for explanation of the second addition way in a modified embodiment. FIG. 17 includes illustrations for explanation of the third addition way in a modified embodiment. FIG. 18 includes illustrations for explanation of the fourth addition way in a modified embodiment. In each of FIG. 15 to FIG. 18, the section “A” illustrates a plurality of steps to produce a first product with a specification A, a plurality of main facilities respectively associated with the steps, and a CO₂ emission amount in each of the steps. The section “B” illustrates a plurality of steps to produce a second product with a specification B, a plurality of main facilities respectively associated with the steps, and a CO₂ emission amount in each of the steps. The section “C” illustrates a plurality of steps to produce a third product with a specification C, a plurality of main facilities respectively associated with the steps, and a CO₂ emission amount in each of the steps. Each of FIG. 15E, FIG. 16D, FIG. 17D, and FIG. 18D includes illustrations of a plurality of steps to produce a fifth product with the specification C, a plurality of main facilities respectively associated with the steps, and a CO₂ emission amount in each of the steps. Each of FIG. 15 to FIG. 18 further illustrates a raw material and a total raw material CO₂ emission amount, defines a transport process or dispatch, which serves as an example predetermined process executed to the product after finish of production of the product to delivery of the product to a delivery destination, as one step, and illustrates a total amount of the product and a total product CO₂ emission amount about the product. FIG. 15D illustrates a total amount of the fourth product with the specification C and a total product CO₂ emission amount about the fourth product.

In FIG. 15 to FIG. 18, the third product serves as a target product of a target type, and the fifth product results in a rejected lot object having a defect after finish of a second step STe-2 among first to fourth steps STe-1 to STe-4 and defined as an unavailable product and removed without dispatch.

In FIG. 15A to FIG. 18A, the first product made of a material “a” with the specification A is dispatched in a transport step STa-S after passing through the first step STa-l executed by a facility 1 as a main facility, the second step STa-2 executed by a facility 2 as a main facility, the third step STa-3 executed by a facility 3 as a main facility, and the fourth step STa-4 executed by a facility 5 as a main facility. Respective step CO₂ emission amounts about the first to fourth steps STa-1 to STa-4 indicate “2 [tCO2]”, “1 [tCO2]”, “3 [tCO2]”, and “3 [tCO2]”. A total raw material CO₂ emission amount thereof indicates “6 [tCO2]”, a total amount of the first product indicates “3 [ton]”, and a total product CO₂ emission amount about the first product reaches “15 [tCO2]”.

In FIG. 15B to FIG. 18B, the second product made of a material “b” with the specification B is dispatched in a transport step STb-S after passing through a first step STb-1 executed by the facility 1 as the main facility, a second step STb-2 executed by the facility 2 as the main facility, a third step STb-3 executed by a facility 4 as a main facility, and a fourth step STb-4 executed by a facility 6 as a main facility. Respective step CO₂ emission amounts about the first to fourth steps STb-1 to STb-4 indicate “2 [tCO2]”, “1 [tCO2]”, “2 [tCO2]”, and “1 [tCO2]”. A total raw material CO₂ emission amount thereof indicates “4 [ton]”, a total amount of the second product indicates “2 [ton]”, and a total product CO₂ emission amount about the second product reaches “10 [tCO2]”.

In FIG. 15C to FIG. 18C, the third product made of the raw material “b” with the specification C is dispatched in a transport step STa-S after passing through a first step STc-1 executed by the facility 1 as the main facility, a second step STc-2 executed by the facility 3 as the main facility, a third step STc-3 executed by the facility 4 as the main facility, and a fourth step STc-4 executed by the facility 5 as the main facility. Respective step CO₂ emission amounts about the first to fourth steps STc-1 to STc-4 indicate “3 [tCO2]”, “2 [tCO2]”, “4 [tCO2]”, and “2 [tCO2]”. A total amount of the third product reaches “5 [ton]”. A total raw material CO₂ emission amount thereof indicates “8 [tCO2]”, a total amount of the third product indicates “5 [ton]”, and a total product CO₂ emission amount about the third product reaches “19 [tCO2]”.

FIG. 15D omits illustration of steps related to the fourth product with the specification C, but illustrates a total amount of the product “2.5 [ton]” and a total product CO₂ emission amount “8 [tCO2]” about the fourth product.

Normally, the fifth product made of the raw material “b” with the specification C in each of FIGS. 15E, 16D, FIG. 17D, and FIG. 18D is dispatched in a transport step STe-S after passing through the first step STe-1 executed by the facility 1 as the main facility, the second step STe-2 executed by the facility 3 as the main facility, the third step STe-3 executed by the facility 4 as the main facility, and the fourth step STe-4 executed by the facility 5 as the main facility. In this example, however, the product results in a rejected lot object having a defect after finish of the second step STe-2 and thus defined as an unavailable product and removed without dispatch. Respective step CO₂ emission amounts about the first and second steps STe-1 and STe-2 indicate “1 [tCO2]” and “2 [tCO2]”. A total raw material CO₂ emission amount thereof indicates “3 [tCO2]”.

The specification A to the specification C differ from one another. The facility 1 to the facility 6 differ from one another. Basically, a value of each CO₂ emission amount represents a numerical value before addition. An amount followed by the arrow sign “⇒” represents a numerical value after the addition in each drawing.

First of all, the first addition way will be described. The first addition way focuses on a type of product, and incorporation of a total rejected lot object CO₂ emission amount about a rejected lot object. In the above-described embodiment, such a type of product is classified depending on a kind or specification, and a consumer. Here, a kind of product is focused on. A carbon dioxide emission amount calculation system (a carbon dioxide emission amount calculation apparatus serving as one example of the system) S adopting the first addition way further operatively includes a rejected lot object emission amount calculation part 61 and an emission amount addition calculation part 62a, each denoted by a dashed-line box in FIG. 1, in a control processor 4.

The rejected lot object emission amount calculation part 61 calculates a total rejected lot object CO₂ emission amount about a rejected lot object.

In the modified embodiment, the raw material emission amount calculation part 42 further calculates, on the basis of a given first unit CO₂ emission amount per unit about a raw material in connection with production leading to the rejected lot object and a total amount of the raw material resulting in the rejected lot object produced in the predetermined period, a total raw material CO₂ emission amount about the raw material in connection with the production leading to the rejected lot object. In the example in FIG. 15E, the raw material resulting in the rejected lot object is the raw material “b”, and the total raw material CO₂ emission amount about the raw material “b” indicates “3 [tCO2]”.

In the modified embodiment, the step emission amount calculation part 44 further calculates, for one or more main facilities associated with one or more steps executed until the removal among a plurality of steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object. In the example in FIG. 15E, the facility 1 is adopted as the main facility in the first step STe-1 and a step CO₂ emission amount about the first step STe-1 executed by the facility 1 indicates “1 [tCO2]”, and the facility 3 is adopted as the main facility in the second step STe-2 and a step CO₂ emission amount about the second step STe-2 executed by the facility 3 indicates “2 [tCO2]”.

Specifically, the rejected lot object emission amount calculation part 61 calculates, as the total rejected lot object CO₂ emission amount, a sum of: one or more total step CO₂ emission amounts about each step executed by each main facility as calculated by the step emission amount calculation part 44 for the one or more main facilities associated with the one or more steps executed until the removal among the steps in the production leading to the rejected lot object; and the total raw material CO₂ emission amount about the raw material in connection with the production leading to the rejected lot object as calculated by the raw material emission amount calculation part 42. In the example in FIG. 15E, the sum “6 [tCO2]” (=3+3) of: a total “3 [tCO2]” (=1+2) of the step CO₂ emission amount “1 [tCO2]” about the first step STe-1 and the step CO₂ emission amount “2 [tCO2]” about the second step STe-2; and the total raw material CO₂ emission amount “3 [tCO2]” about the second raw material “b” is obtained.

The emission amount addition calculation part 62a adds a predetermined proportion (A-th proportion) of the total rejected lot object CO₂ emission amount calculated by the rejected lot object emission amount calculation part 61 to the total target product CO₂ emission amount calculated by the target product emission amount calculation part 49 when the target product of the target type has a kind which is identical to a kind of the rejected lot object. The A-th proportion represents a proportion of a total amount of the target product of the target type with respect to a total amount of a product (including the target product of the target type) having a kind which is identical to the kind of the rejected lot object and produced in the predetermined period. In the example in FIG. 15, the third product with the specification C in FIG. 15C serves as the target product of the target type. It is seen from this perspective that the kind (specification) C of the target product is identical to the kind (specification) C of the fifth product being the rejected lot object in FIG. 15E. Hence, the emission amount addition calculation part 62a firstly obtains a total amount “7.5 [ton]” (=5+2.5) of the product (i.e., each of the third and fourth products) having the kind C which is identical to the kind C of the fifth product being the rejected lot object and produced in the predetermined period and obtains the total amount “5 [ton]” of the third product serving as the target product of the target type to calculate the A-th proportion, and finally obtains the A-th proportion “5/7.5”. The emission amount addition calculation part 62a then adds only a portion “6×(5/7.5)=4” being the A-th proportion “5/7.5” of the total rejected lot object CO₂ emission amount “6 [tCO2]” to the total target product CO₂ emission amount “19 [tCO2]” (=8+3+2+4+2) obtained by the target product emission amount calculation part 49. A total target product CO₂ emission amount about the third product after the addition reaches “23 [tCO2]” (=19+4). In a case where the fourth product serves as a target product of a target type, a remaining amount “2 [tCO2]” amount of the total rejected lot object CO₂ emission amount “6 [tCO2]” serves as a portion “6×(2.5/7.5)”=“2” being the A-th proportion “2.5/7.5” of the total rejected lot object CO₂ emission amount “6 [tCO2]” and is added to the total CO₂ emission amount “8 [tCO2]” about the fourth product. A total CO₂ emission amount about the fourth product after the addition reaches “10 [tCO2]” (=8+2).

According to the first addition way, the A-th proportion may represent a proportion of a total target product CO₂ emission amount about the target product of the target type with respect to a total product CO₂ emission amount about a product (including the target product of the target type) having a kind which is identical to the kind of the rejected lot object and produced in the predetermined period in connection with the product. For instance, the A-th proportion related to the third product results in “19 /(9+8)=19/27”.

The carbon dioxide emission amount calculation apparatus S enables incorporation of such a rejected lot object CO₂ emission amount about a rejected lot object. Specifically, the apparatus enables incorporation of the rejected lot object CO₂ emission amount into the total target product CO₂ emission amount, when a type of product is classified depending on a kind and a consumer and the target product of the target type has a kind which is identical to the kind of the rejected lot object.

As described above, each type of product is classified depending on a kind or specification and a consumer or orderer. Accordingly, at receiving of a plurality of orders at different times for the same kind from the same consumer, the same product number is given to these orders and products produced in response to the orders are collected to one group. Instead, such a type of product may be classified depending on a kind or specification. In this case, the emission amount addition calculation part 62a adds a predetermined proportion (A′-th proportion) of the total rejected lot object CO₂ emission amount calculated by the rejected lot object emission amount calculation part 61 to the total target product CO₂ emission amount calculated by the target product emission amount calculation part 49 when the target type is identical to a type of the rejected lot object. The A′-th proportion represents a proportion of the total amount of the target product of the target type with respect to a total amount of a product (including the target product of the target type) having a type which is identical to the type of the rejected lot object and produced in the predetermined period. The A′-th proportion may represent a proportion of the total target product CO₂ emission amount about the target product of the target type with respect to a total product CO₂ emission amount about a product having a kind which is identical to the kind of the rejected lot object and produced in the predetermined period. The rejected lot object emission amount calculation part 61, a raw material emission amount calculation part 42, and a step emission amount calculation part 44 respectively operate in the same manner as those described above. The carbon dioxide emission amount calculation apparatus S enables incorporation of such a rejected lot object CO₂ emission amount about a rejected lot object. Specifically, the apparatus enables incorporation of the rejected lot object CO₂ emission amount into the total target product CO₂ emission amount, when a type of product is classified depending on a kind or specification and a type of the target product of the target type is identical to the type of the rejected lot object.

Next, the second addition way will be described. The second addition way focuses on a raw material for a product, and incorporation of a total rejected lot object CO₂ emission amount about a rejected lot object. A carbon dioxide emission amount calculation system (a carbon dioxide emission amount calculation apparatus serving as one example of the system) S adopting the second addition way further operatively includes a rejected lot object emission amount calculation part 61 and an emission amount addition calculation part 62b, each denoted by a dashed-line box in FIG. 1, in a control processor 4. The rejected lot object emission amount calculation part 61, a raw material emission amount calculation part 42, and a step emission amount calculation part 44 in the carbon dioxide emission amount calculation apparatus S adopting the second addition way are respectively similar to the rejected lot object emission amount calculation part 61, the raw material emission amount calculation part 42, and the step emission amount calculation part 44 in the carbon dioxide emission amount calculation apparatus S adopting the first addition way, and thus, description therefor will be omitted here.

The emission amount addition calculation part 62b adds a predetermined proportion (B-th proportion) of the total rejected lot object CO₂ emission amount calculated by the rejected lot object emission amount calculation part 61 to the total target product CO₂ emission amount calculated by the target product emission amount calculation part 49 when the material for the target product of the target type is identical to a raw material for the rejected lot object. The B-th proportion represents a proportion of a total raw material CO₂ emission amount about the raw material for the target product of the target type with respect to a total raw material CO₂ emission amount about a raw material for a product (including the target product of the target type) made of a raw material which is identical to the raw material for the rejected lot object and produced in the predetermined period. In the example in FIG. 16, the third product with the specification C in FIG. 16C serves as the target product of the target type. In this respect, the raw material “b” for the target product is identical to the raw material “b” for the fifth product being a rejected lot object. Hence, the emission amount addition calculation part 62b firstly obtains the total raw material CO₂ emission amount “12 [tCO2]” (=4+8) about the raw material for the product (including target product of the target type) made of the raw material “b” which is identical to the raw material “b” for the rejected lot object and being a product (i.e., each of the second and third products) produced in the predetermined period and obtains the total raw material CO₂ emission amount “8 [tCO2]” about the raw material for the third product serving as the target product of the target type to calculate the B-th proportion, and finally obtains the B-th proportion “8/12=2/3”. The emission amount addition calculation part 62b then adds only a portion “6×(2/3)”=“4” being the B-th proportion “2/3” of the total rejected lot object CO₂ emission amount “6 [tCO2]” to the total target product CO₂ emission amount “19 [tCO2]”(=8+3+2+4+2) obtained by the target product emission amount calculation part 49. A total target product CO₂ emission amount about the third product after the addition reaches “23 [tCO2]”. Here, in FIG. 16C, only the portion “6×(2/3)”=“4” being the B-th proportion “2/3” of the total rejected lot object CO₂ emission amount “6 [tCO2]” is added to the total raw material CO₂ emission amount “8 [tCO2]” about the raw material in connection with the production of the third product. In this regard, the total target product CO₂ emission amount about the third product incorporates the total raw material CO₂ emission amount about the raw material in connection with the production of the third product. Hence, the same result is acquired even by adding the portion to any of the total emission amounts. In a case where the second product serves as a target product of a target type, a remaining amount “2 [tCO2]” of the total rejected lot object CO₂ emission amount “6 [tCO2]” to serve as a portion “6×(1/3)”=“2” being the B-th proportion “1/3” of the total rejected lot object CO₂ emission amount “6 [tCO2]” is added to the total CO₂ emission amount “10 [tCO2]” about the second product. A total CO₂ emission amount about the second product after the addition reaches “12 [tCO2]”.

According to the second addition way, the B-th proportion may represent a proportion of a total amount of the raw material for the target product of the target type with respect to the total amount of the raw material for the product (including the target product of the target type) made of the raw material which is identical to the raw material for the rejected lot object and produced in the predetermined period.

The carbon dioxide emission amount calculation apparatus S enables incorporation of such a rejected lot object CO₂ emission amount about a rejected lot object. Specifically, the apparatus enables incorporation of the rejected lot object CO₂ emission amount into the total target product CO₂ emission amount when the raw material for the target product is identical to the raw material for the rejected lot object CO₂ emission amount.

Moreover, the third addition way will be described. The third addition way focuses on a facility that executes a step, and incorporation of a total rejected lot object CO₂ emission amount about a rejected lot object. A carbon dioxide emission amount calculation system (a carbon dioxide emission amount calculation apparatus serving as one example of the system) S adopting the third addition way further operatively includes a rejected lot object emission amount calculation part 61 and an emission amount addition calculation part 62c, each denoted by a dashed-line box in FIG. 1, in a control processor 4. The rejected lot object emission amount calculation part 61, a raw material emission amount calculation part 42, and a step emission amount calculation part 44 in the carbon dioxide emission amount calculation apparatus S adopting the third addition way are respectively similar to the rejected lot object emission amount calculation part 61, the raw material emission amount calculation part 42, and the step emission amount calculation part 44 in the carbon dioxide emission amount calculation apparatus S adopting the first addition way, and thus, description therefor will be omitted here.

The emission amount addition calculation part 62c adds, when a plurality of main facilities respectively associated with a plurality of steps in production of a target product of a target type include one or more main facilities which are the same as one or more main facilities associated with one or more steps executed until the removal among a plurality of steps in production leading to the rejected lot object, a predetermined proportion (C-th proportion) of a total rejected lot object CO₂ emission amount calculated by the rejected lot object emission amount calculation part 61 to a step CO₂ emission amount about each step executed by each main facility in connection with the production of the target product of the target type as calculated by the step emission amount calculation part 44, for the same main facility. The C-th proportion represents, for the same main facility, a proportion of a step CO₂ emission amount about a step executed by the main facility in connection with the production of the target product of the target type calculated by the step emission amount calculation part 44 with respect to a total step CO₂ emission amount about the step executed by the main facility as calculated by the step emission amount calculation part 44 in connection with a product (including the target product of the target type) produced in the predetermined period. Specifically, in existence of a plurality of types for the same main facility, a step CO₂ emission amount about the step executed by the main facility of a specific type calculated by the step emission amount calculation part 44 in connection with the product (including the target product of the target type) produced in the predetermined period is calculated for each of the types. A total of respective step CO₂ emission amounts calculated for the plurality of types is calculated. For each of the types, a proportion of the step CO₂ emission amount about the step executed by the main facility of the type in connection with the production of the target product of the target type as calculated by the step emission amount calculation part 44 with respect to the total is calculated as the C-th proportion about the main facility of the type.

In the example in FIG. 17, the third product with the specification C in FIG. 17C serves as the target product of the target type. Main facilities respectively associated with the first to fourth steps STc-1 to STc-4 in the production of the target product include the facility 1, the facility 3, the facility 4, and the facility 5. Main facilities associated with the first and second steps STe-1 and STe-2 executed until the removal among the first to fourth steps STe-1 to STe-4 in the production leading to the fifth product being the rejected lot object include the facility 1 and the facility 3. Thus, each of the facility 1 and the facility 3 is common in the production of the target product and the production leading to the rejected lot object as the same facility. The facility 1 is adopted in each of the first step STa-1 related to the first product, the first step STb-1 related to the second product, and the first step STc-1 related to the third product. Step CO₂ emission amounts about these steps respectively indicate “2 [tCO2]”, “2 [tCO2]”, and “3 [tCO2]”. The facility 3 is adopted in each of the third step STa-3 related to the first product and the second step STc-2 related to the third product. Step CO₂ emission amounts about these steps respectively indicate “3 [tCO2]” and “2 [tCO2]”. To calculate the C-th proportion, the emission amount addition calculation part 62c firstly obtains: the total step emission amount “12” (=2+2+3+3+2) [tCO2]” about each step executed by the main facility as calculated by the step emission amount calculation part in connection with the product produced in the predetermined period, for the same main facility; the step CO₂ emission amount “3 [tCO2]” about the step executed by the facility 1 in connection with the production of the third product serving as the target product of the target type for the facility 1 and the C-th proportion “3/12” about the facility 1; and the step CO₂ emission amount “2 [tCO2]” about the step executed by the facility 3 in connection with the production of the third product and the C-th proportion “2/12” about the facility 3. The emission amount addition calculation part 62c then adds only a portion “6×(3/12)”=“1.5” being the C-th proportion “3/12” of the total rejected lot object CO₂ emission amount “6 [tCO2]” to the step CO₂ emission amount “3 [tCO2]” about the step executed by the facility 1 in connection with the production of the third product for the facility 1, and adds only a portion “6×(2/12)”=“1” being the C-th proportion “2/12” of the total rejected lot object CO₂ emission amount “6 [tCO2]” to the step CO₂ emission amount “2 [tCO2]” about the step executed by the facility 1 in connection with the production of the third product for the facility 3. Regarding the third product after the addition, the step CO₂ emission amount about the first step STc-1 reaches “4.5 [tCO2]” and the step CO₂ emission amount about the second step STc-2 reaches “3 [tCO2]”. A total target product CO₂ emission amount about the third product after the addition reaches “21.5 [tCO2]”. In a case where each of the first and second products serves as a target product of a target type, a remaining amount “4.5 [tCO2]” of the total rejected lot object CO₂ emission amount “6 [tCO2]” may be added to the relevant emission amount.

According to the third addition way, the C-th proportion may represent, for the same main facility, a proportion of a total production load amount required in the main facility in the predetermined period in connection with the production of the target product of the target type with respect to a total production load required in the main facility in the predetermined period in connection with the production of the product (including the target product of the target type) produced in the predetermined period.

The carbon dioxide emission amount calculation apparatus S enables incorporation of such a rejected lot object CO₂ emission amount about a rejected lot object. Specifically, when the main facilities related to the target product include a main facility which is the same as a main facility related to the rejected lot object, the apparatus enables, for the same facility, incorporation of the rejected lot object CO₂ emission amount into the step CO₂ emission amount about the step executed by the main facility in connection with the production of the target product of the target type.

Furthermore, the fourth addition way will be described. The fourth addition way focuses on a facility that executes a step, and incorporation of a step CO₂ emission amount about a certain step executed by an associated facility in connection with production leading to a rejected lot object for the facility. A carbon dioxide emission amount calculation system (a carbon dioxide emission amount calculation apparatus serving as one example of the system) S adopting the fourth addition way further operatively includes an emission amount addition calculation part 62d, denoted by a dashed-line box in FIG. 1, in a control processor 4. A raw material emission amount calculation part 42 and a step emission amount calculation part 44 in the carbon dioxide emission amount calculation apparatus S adopting the fourth addition way are respectively similar to the raw material emission amount calculation part 42 and the step emission amount calculation part 44 in the carbon dioxide emission amount calculation apparatus S adopting the first addition way, and thus, description therefor will be omitted here.

The emission amount addition calculation part 62d adds, when a plurality of main facilities respectively associated with a plurality of steps in production of a target product of a target type include one or more main facilities which are the same as one or more main facilities associated with one or more steps executed until the removal among a plurality of steps in the production leading to the rejected lot object, a predetermined first proportion of a step CO₂ emission amount about each step executed by each main facility in connection with the production leading to the rejected lot object as calculated by the step emission amount calculation part 44 to a step CO₂ emission amount about the step executed by the main facility in connection with the production of the target product of the target type as calculated by the step emission amount calculation part 44, for the same main facility. The first proportion represents a proportion of a step CO₂ emission amount about a step executed by the main facility in connection with the production of the target product of the target type calculated by the step emission amount calculation part with respect to a total step CO₂ emission amount about the step executed by the main facility as calculated by the step emission amount calculation part in connection with a product (including the target product of the target type) produced in the predetermined period. Specifically, in existence of a plurality of types for the same main facility, a total step CO₂ emission amount about each step executed by a main facility of a specific type as calculated by the step emission amount calculation part 44 in connection with the product (including the target product of the target type) produced in the predetermined period is calculated for each of the types. A proportion of the step CO₂ emission amount about the step executed by the main facility of the type in connection with the production of the target product of the target type as calculated by the step emission amount calculation part 44 with respect to the total amount is calculated as the first proportion. That is to say, according to the fourth addition, for each type of facility, only the predetermined first proportion of the step CO₂ emission amount about the step executed by the main facility of the type in connection with the production leading to the rejected lot object is added.

In the example in FIG. 18, the third product with the specification C in FIG. 18C serves as the target product of the target type. Main facilities respectively associated with the first to fourth steps STc-1 to STc-4 in the production of the target product include the facility 1, the facility 3, the facility 4, and the facility 5. The main facilities associated with the first and second steps STe-1 and STe-2 executed until the removal among the first to fourth steps STe-1 to STe-4 in the production leading to the fifth product being the rejected lot object include the facility 1 and the facility 3. Thus, each of the facility 1 and the facility 3 is common in the production of the target product and the production leading to the rejected lot object as the same facility. The facility 1 is adopted in each of the first step STa-1 related to the first product, the first step STb-1 related to the second product, and the first step STc-1 related to the third product. Step CO₂ emission amounts about these steps respectively indicate “2 [tCO2]”, “2 [tCO2]”, and “3 [tCO2]”. The facility 3 is adopted in each of the third step STa-3 related to the first product and the second step STc-2 related to the third product. Step CO₂ emission amounts about these steps respectively indicate “3 [tCO2]” and “2 [tCO2]”. For the facility 1, the emission amount addition calculation part 62d firstly obtains the total step CO₂ emission amount “7 (=2+2+1) [tCO2]” about each step executed by the facility 1 as calculated by the step emission amount calculation part 44 in connection with the product produced in the predetermined period and the step CO₂ emission amount “3 [tCO2]” about the step executed by the facility 1 in connection with the production of the third product serving as the target product of the target type to calculate the first proportion, and obtains the first proportion “3/7” about the facility 1, and then, adds only a portion “1×(3/7)”=“0.43” (rounded off at the third decimal place, this is applicable hereafter) being the first proportion “3/7” about the facility 1 of the step CO₂ emission amount “1 [tCO2]” about the step executed by the facility 1 in connection with the production leading to the rejected lot object to the step CO₂ emission amount “3 [tCO2]” about the step executed by the facility 1 in connection with the production of the third product. For the facility 3, the emission amount addition calculation part 62d subsequently obtains the total step CO₂ emission amount “5 (=3+2) [tCO2]” about each step executed by the facility 3 as calculated by the step emission amount calculation part 44 in connection with the product produced in the predetermined period and the step CO₂ emission amount “2 [tCO2]” about the step executed by the facility 3 in connection with the production of the third product serving as the target product of the target type to calculate the first proportion, and obtains the first proportion “2/5” about the facility 3, and then, adds only a portion “2×(2/5)”=“0.8” being the first proportion “2/5” about the facility 3 of the step CO₂ emission amount “2 [tCO2]” about the step executed by the facility 3 in connection with the production leading to the rejected lot object to the step CO₂ emission amount “2 [tCO2]” about the step executed by the facility 3 in connection with the production of the third product. Regarding the third product after the addition, the step CO₂ emission amount about the first step STc-1 reaches “3.43 [tCO2]” and the step CO₂ emission amount about the second step STc-2 reaches “2.8 [tCO2]”. A total target product CO₂ emission amount about the third product after the addition reaches “20.23 [tCO2]”. It is noted here that the total emission amount does not incorporate a raw material CO₂ emission amount about a raw material in connection with the production leading to the rejected lot object to be added thereto. The addition will be described later. In a case where each of the first and second products serves as a target product of a target type, a remaining amount “4/7 [tCO2]” of the step CO₂ emission amount “1 [tCO2]” about the step executed by the facility 1 in connection with the production leading to the rejected lot object is added to the relevant emission amount. In a case where the first product serves as a target product of a target type, a remaining amount “1.2 [tCO2]” of the step CO₂ emission amount “2 [tCO2]” about the step executed by the facility 3 in connection with the production leading to the rejected lot object is added to the relevant emission amount.

According to the fourth addition way, the first proportion may represent a proportion of a total production load amount required in the main facility in the predetermined period in connection with the production of the target product of the target type with respect to a total production load required in the main facility in the predetermined period in connection with the production of the product (including the target product of the target type) produced in the predetermined period.

The emission amount addition calculation part 62d further adds a predetermined second proportion of the total raw material CO₂ emission amount about the raw material in connection with the production leading to the rejected lot object as calculated by the raw material emission amount calculation part 42 to the total target product CO₂ emission amount calculated by the target product emission amount calculation part 49, when the product has a type classified depending on a kind and a consumer and the kind of the target product of the target type is identical to a kind of the rejected lot object. The second proportion represents a proportion of a total amount of the target product of the target type with respect to a total amount of a product (including the target product of the target type) having a kind which is identical to the kind of the rejected lot object and produced in the predetermined period. The second proportion may represent a proportion of the total target product CO₂ emission amount about the target product of the target type with respect to a total product CO₂ emission amount about a product (including the target product of the target type) having a kind which is identical to the kind of the rejected lot object and produced in the predetermined period. The second proportion is calculatable in the same manner as the A-th proportion in the first addition.

In the example in FIG. 18, the third product with the specification C in FIG. 18C serves as the target product of the target type. It is seen from this perspective that the raw material “b” for the target product is identical to the raw material “b” for the fifth product being a rejected lot object. Hence, the emission amount addition calculation part 62d firstly obtains the total product CO₂ emission amount “29 [ton]” (=10+19) about a product (including the target product of the target type) made of the raw material “b” which is identical to the raw material “b” for the rejected lot object and being a product (i.e., each of the second and third products) produced in the predetermined period and obtains the total target product material CO₂ emission amount “19 [tCO2]”(=8+3+2+4+2) about the target product of the target type in connection with the third product serving as the target product of the target type to calculate the second proportion, and finally obtains the second proportion “19/29”. The emission amount addition calculation part 62d then adds only a portion “3×(19/29)”=“1.97” being the second proportion “19/29” of a total raw material CO₂ emission amount “3 [tCO2]” about the raw material in connection with the production leading to the rejected lot object as calculated by the raw material emission amount calculation part 42 to the total target product CO₂ emission amount “19 [tCO2]” calculated by the target product emission amount calculation part 49. A total target product CO₂ emission amount about the third product after the addition reaches “22.2 [tCO2]” (=20.23+1.97). In a case where each of the first and second products serves as a target product of a target type, a remaining amount “3×(10/29) [tCO2]” of the total raw material CO₂ emission amount “3 [tCO2]” about the raw material in connection with the production leading to the rejected lot object is added to the relevant emission amount.

According to the fourth addition way, the emission amount addition calculation part 62d may further add a predetermined third portion of the total raw material CO₂ emission amount about the raw material in connection with the production leading to the rejected lot object as calculated by the raw material emission amount calculation part 42 to the total target product CO₂ emission amount calculated by the target product emission amount calculation part 49 when the target type is identical to a type of the rejected lot object. Alternatively, the emission amount addition calculation part 62d may further add a predetermined fourth proportion of the total raw material CO₂ emission amount about the raw material in connection with the production of the rejected lot object as calculated by the raw material emission amount calculation part 42 to the total target product CO₂ emission amount calculated by the target product emission amount calculation part 49 when the raw material for the target product of the target type and the raw material for the rejected lot object are identical to each other. The third proportion may represent a proportion of the total amount of the target product of the target type with respect to a total amount of a product (including the target product of the target type) having a type which is identical to the type of the rejected lot object and produced in the predetermined period, or may represent a proportion of the total target product CO₂ emission amount about the target product of the target type with respect to a total product CO₂ emission amount about a product having a kind which is identical to the kind of the rejected lot object and produced in the predetermined period. The third proportion is obtainable in the same manner as the A′-th proportion in the first addition. The fourth proportion may represent a proportion of the total amount of the target product of the target type with respect to a total amount of a product (including the target product of the target type) made of a material which is identical to the raw material for the rejected lot object and produced in the predetermined period, or may represent a proportion of a total raw material CO₂ emission amount about the raw material for the target product of the target type with respect to a total raw material CO₂ emission amount about a raw material for the product made of the raw material which is identical to the raw material for the rejected lot object and produced in the predetermined period. The fourth proportion is calculatable in the same manner as the B-th proportion in the second addition.

The carbon dioxide emission amount calculation apparatus S enables incorporation of such a rejected lot object CO₂ emission amount about a rejected lot object. Specifically, when the main facilities related to the target product include a main facility which is the same as a main facility related to the rejected lot object, the apparatus enables incorporation of a step CO₂ emission amount about the step executed by the main facility in connection with the production leading to the rejected lot object into the step CO₂ emission amount about the step executed by the main facility in connection with the production of the target product of the target type for the same main facility.

The raw material may include a portion to remain as so-called processing wastes, e.g., shavings and chips, without being made into a product in a certain step. In the existence of the processing wastes, a CO₂ emission amount about the processing wastes is incorporated into a step CO₂ emission amount about the step related to the processing wastes. Accordingly, the first or fourth addition way is adoptable.

This application is based on Japanese Patent Application No. 2022-193236 field on Dec. 2, 2022 and Japanese Patent Application No. 2023-4652 field on Jan. 16, 2023 in Japan Patent Office, and includes contents thereof.

Although the present invention has been fully described by way of the embodiments and examples with reference to the above-described specific examples, it is to be understood that various changes and/or modifications to the embodiments and examples will be apparent to those skilled in the art. Therefore, unless otherwise such changes and modifications to be made by those skilled in the art depart from the scope of the present invention hereinafter defined, they should be construed as being included therein.

### INDUSTRIAL APPLICABILITY

The present invention provides a carbon dioxide emission amount calculation system and a carbon dioxide emission amount calculation method to calculate an emission amount of carbon dioxide emitted in relation to a product of a predetermined type to be produced in a predetermined period.

1. A carbon dioxide emission amount calculation system that calculates CO₂ emission amounts about a target product of a target type in a plant configured to produce a certain product of a plurality of types through a plurality of steps, the carbon dioxide emission amount calculation system comprising:
a raw material emission amount calculation part;
a main facility unit emission amount calculation part; and
a step emission amount calculation part, wherein
the raw material emission amount calculation part calculates, on the basis of a first unit CO₂ emission amount per unit about a raw material in connection with production of the target product of the target type and a total amount of the raw material used for the target product of the target type produced in a predetermined period, a total raw material CO₂ emission amount about the raw material in connection with the production of the target product of the target type,
the main facility unit emission amount calculation part calculates, for each of a plurality of main facilities respectively associated with the steps in the production of the target product of the target type, a second unit CO₂ emission amount per unit about each of the main facilities on the basis of a total production load amount required in the main facility associated with the step to execute the step in the production in the predetermined period and a total CO₂ emission amount about the main facility with respect to the total production load amount, and
the step emission amount calculation part calculates, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated by the main facility unit emission amount calculation part and the total production load amount required in the main facility in the production in the predetermined period in connection with the production of the target product of the target type.

2. The carbon dioxide emission amount calculation system according to claim 1, further comprising
a target product emission amount calculation part that calculates, as a total target product CO₂ emission amount about the target product of the target type, a sum of: a total of respective step CO₂ emission amounts calculated by the step emission amount calculation part about the steps executed by the main facilities for each of the main facilities respectively associated with the steps in the production of the target product of the target type; and the total raw material CO₂ emission amount about the raw material calculated by the raw material emission amount calculation part in connection with the production of the target product of the target type.

3. The carbon dioxide emission amount calculation system according to claim 1, further comprising
an emission allotment amount calculation part that calculates, concerning an auxiliary facility indirectly involved in the production of the product via a specific main facility and being adoptable for a plurality of facilities, a CO₂ emission allotment amount about a certain facility that utilizes the auxiliary facility by allotting a total auxiliary facility CO₂ emission amount about the auxiliary facility in the predetermined period to each of the facilities that utilizes the auxiliary facility at a predetermined proportion, wherein,
when the main facility has an association with the auxiliary facility in the calculation of the second unit CO₂ emission amount per unit about the main facility, the main facility emission amount calculation part calculates a new total CO₂ emission amount about the main facility with respect to the total production load amount by adding the CO₂ emission allotment amount about the facility corresponding to the main facility calculated by the emission allotment amount calculation part to the total CO₂ emission amount about the main facility with respect to the total production load amount, and calculates the second unit CO₂ emission amount per unit about the main facility on the basis of the calculated new total CO₂ emission amount about the main facility with respect to the total production load amount.

4. The carbon dioxide emission amount calculation system according to claim 1, further comprising
an accompanying process emission amount calculation part that calculates, on the basis of a third unit CO₂ emission amount per unit about a predetermined process executed to the product after the production of the product to delivery of the product to a delivery destination and a total amount of the target product of the target type produced in the plant in the predetermined period, a total accompanying process CO₂ emission amount about the predetermined process to the target product of the target type.

5. The carbon dioxide emission amount calculation system according to claim 1, wherein the plant bears a CO₂ emission source that emits CO₂, the CO₂ emission source including the main facility, an auxiliary facility, a predetermined process, and a remaining emission source remaining except the main facility, the auxiliary facility, and the predetermined process, the carbon dioxide emission amount calculation system further comprising:
a first remaining emission amount calculation part that calculates a fourth unit CO₂ emission amount per product unit about the remaining emission source on the basis of a total remaining CO₂ emission amount about the remaining emission source in the predetermined period and a total amount of the product produced in the plant in the predetermined period; and
a second remaining emission amount calculation part that calculates a total second remaining CO₂ emission amount about the remaining emission source in connection with the production of the target product of the target type on the basis of the fourth unit CO₂ emission amount per product unit about the remaining emission source calculated by the first remaining emission amount calculation part and a total amount of the target product of the target type produced in the predetermined period.

6. The carbon dioxide emission amount calculation system according to claim 2, wherein the product has a type classified depending on a kind and a consumer, the carbon dioxide emission amount calculation system further comprising:
a rejected lot object emission amount calculation part that calculates a total rejected lot object CO₂ emission amount about a rejected lot object to be removed after finish of a certain step of the steps; and
an emission amount addition calculation part that adds a predetermined proportion of the total rejected lot object CO₂ emission amount calculated by the rejected lot object emission amount calculation part to the total target product CO₂ emission amount calculated by the target product emission amount calculation part when the target product of the target type has a kind which is identical to a kind of the rejected lot object, wherein
the raw material emission amount calculation part further calculates, on the basis of a first unit CO₂ emission amount per unit about a raw material in connection with production leading to the rejected lot object and a total amount of the raw material resulting in the rejected lot object produced in the predetermined period, a total raw material CO₂ emission amount about the raw material in connection with the production leading to the rejected lot object,
the step emission amount calculation part further calculates, for one or more main facilities associated with one or more steps executed until the removal among a plurality of steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object, and
the rejected lot object emission amount calculation part calculates, as the total rejected lot object CO₂ emission amount, a sum of: one or more total step CO₂ emission amounts about each step executed by each main facility as calculated by the step emission amount calculation part for the one or more main facilities associated with the one or more steps executed until the removal among the steps in the production leading to the rejected lot object; and the total raw material CO₂ emission amount about the raw material in connection with the production leading to the rejected lot object as calculated by the raw material emission amount calculation part.

7. The carbon dioxide emission amount calculation system according to claim 2, further comprising:
a rejected lot object emission amount calculation part that calculates a total rejected lot object CO₂ emission amount about a rejected lot object to be removed after finish of a certain step of the steps; and
an emission amount addition calculation part that adds a predetermined proportion of the total rejected lot object CO₂ emission amount calculated by the rejected lot object emission amount calculation part to the total target product CO₂ emission amount calculated by the target product emission amount calculation part when the target type is identical to a type of the rejected lot object, wherein
the raw material emission amount calculation part further calculates, on the basis of a first unit CO₂ emission amount per unit about a raw material in connection with production leading to the rejected lot object and a total amount of the raw material resulting in the rejected lot object produced in the predetermined period, a total raw material CO₂ emission amount about the raw material in connection with the production leading to the rejected lot object,
the step emission amount calculation part further calculates, for one or more main facilities associated with one or more steps executed until the removal among a plurality of steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object, and
the rejected lot object emission amount calculation part calculates, as the total rejected lot object CO₂ emission amount, a sum of: one or more total step CO₂ emission amounts about each step executed by each main facility as calculated by the step emission amount calculation part for the one or more main facilities associated with the one or more steps executed until the removal among the steps in the production leading to the rejected lot object; and the total raw material CO₂ emission amount about the raw material in connection with the production leading to the rejected lot object as calculated by the raw material emission amount calculation part.

8. The carbon dioxide emission amount calculation system according to claim 2, further comprising:
a rejected lot object emission amount calculation part that calculates a total rejected lot object CO₂ emission amount about a rejected lot object to be removed after finish of a certain step of the steps; and
an emission amount addition calculation part that adds a predetermined proportion of the total rejected lot object CO₂ emission amount calculated by the rejected lot object emission amount calculation part to the total target product CO₂ emission amount calculated by the target product emission amount calculation part when the raw material for the target product of the target type is identical to a raw material for the rejected lot object, wherein
the raw material emission amount calculation part further calculates, on the basis of a first unit CO₂ emission amount per unit about the raw material in connection with production leading to the rejected lot object and a total amount of the raw material resulting in the rejected lot object produced in the predetermined period, a total raw material CO₂ emission amount about the raw material in connection with the production leading to the rejected lot object,
the step emission amount calculation part further calculates, for one or more main facilities associated with one or more steps executed until the removal among a plurality of steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object, and
the rejected lot object emission amount calculation part calculates, as the total rejected lot object CO₂ emission amount, a sum of: one or more total step CO₂ emission amounts about each step executed by each main facility as calculated by the step emission amount calculation part for the one or more main facilities associated with the one or more steps executed until the removal among the steps in the production leading to the rejected lot object; and the total raw material CO₂ emission amount about the raw material in connection with the production leading to the rejected lot object as calculated by the raw material emission amount calculation part.

9. The carbon dioxide emission amount calculation system according to claim 2, further comprising:
a rejected lot object emission amount calculation part that calculates a total rejected lot object CO₂ emission amount about a rejected lot object to be removed after finish of a certain step of the steps; and
an emission amount addition calculation part that adds, when the main facilities respectively associated with the steps in the production of the target product of the target type include one or more main facilities which are the same as one or more main facilities associated with one or more steps executed until the removal among a plurality of steps in production leading to the rejected lot object, a predetermined proportion of the total rejected lot objection CO₂ emission amount calculated by the rejected lot object emission amount calculation part to a step CO₂ emission amount about each step executed by each main facility in connection with the production of the target product of the target type as calculated by the step emission amount calculation part, for the same main facility, wherein
the raw material emission amount calculation part further calculates, on the basis of a first unit CO₂ emission amount per unit about a raw material in connection with production leading to the rejected lot object and a total amount of the raw material resulting in the rejected lot object produced in the predetermined period, a total raw material CO₂ emission amount about the raw material in connection with the production leading to the rejected lot object,
the step emission amount calculation part further calculates, for the one or more main facilities associated with the one or more steps executed until the removal among the steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object, and
the rejected lot object emission amount calculation part calculates, as the total rejected lot object CO₂ emission amount, a sum of: one or more total step CO₂ emission amounts about each step executed by each main facility as calculated by the step emission amount calculation part for the one or more main facilities associated with the one or more steps executed until the removal among the steps in the production leading to the rejected lot object; and the total raw material CO₂ emission amount about the raw material in connection with the production leading to the rejected lot object as calculated by the raw material emission amount calculation part.

10. The carbon dioxide emission amount calculation system according to claim 2, wherein the raw material emission amount calculation part further calculates, on the basis of a first unit CO₂ emission amount per unit about a raw material in connection with production leading to a rejected lot object to be removed after finish of a specific step of the steps and a total amount of the raw material resulting in the rejected lot object produced in the predetermined period, a total raw material CO₂ emission amount about the raw material in connection with the production leading to the rejected lot object, and
the step emission amount calculation part further calculates, for one or more main facilities associated with one or more steps executed until the removal among a plurality of steps in the production leading to the rejected lot object, a step CO₂ emission amount about a step executed by the main facility in connection with the production leading to the rejected lot object, on the basis of the second unit CO₂ emission amount per unit about the main facility calculated by the main facility unit emission amount calculation part and a total production load amount required in the main facility in the predetermined period in connection with the production leading to the rejected lot object, the carbon dioxide emission amount calculation system further comprising:
an emission amount addition calculation part that adds, when the main facilities respectively associated with the steps in the production of the target product of the target type include the one or more main facilities which are the same as the one or more main facilities associated with the one or more steps executed until the removal among the steps in the production leading to the rejected lot object, a predetermined first proportion of a step CO₂ emission amount about each step executed by each main facility in connection with the production leading to the rejected lot object as calculated by the step emission amount calculation part to a step CO₂ emission amount about the step executed by the main facility in connection with the production of the target product of the target type as calculated by the step emission amount calculation part, for the same main facility, wherein
the emission amount addition calculation part further executes:
adding a predetermined second proportion of the total raw material CO₂ emission amount about the raw material in connection with the production leading to the rejected lot object as calculated by the raw material emission amount calculation part to the total target product CO₂ emission amount calculated by the target product emission amount calculation part, when the product has a type classified depending on a kind and a consumer and the target product of the target type has a kind which is identical to the kind of the rejected lot object; or
adding a predetermined third proportion of the total raw material CO₂ emission amount about the raw material in connection with the production leading to the rejected lot object as calculated by the raw material emission amount calculation part to the total target product CO₂ emission amount calculated by the target product emission amount calculation part when the target type is identical to a type of the rejected lot object; or
adding a predetermined fourth proportion of the total raw material CO₂ emission amount about the raw material in connection with the production of the rejected lot object as calculated by the raw material emission amount calculation part to the total target product CO₂ emission amount calculated by the target product emission amount calculation part when the raw material for the target product of the target type and the raw material for the rejected lot object are identical to each other.

11. A carbon dioxide emission amount calculation method of calculating a CO₂ emission amount about a target product of a target type in a plant configured to produce a certain product of a plurality of types through a plurality of steps, the carbon dioxide emission amount calculation method comprising:
a row material emission amount calculation step;
a main facility unit emission amount calculation step; and
a step emission amount calculation step, wherein
the raw material emission amount calculation step includes calculating, on the basis of a first unit CO₂ emission amount per unit about a raw material in connection with production of the target product of the target type and a total amount of the raw material used for the target product of the target type produced in a predetermined period, a total raw material CO₂ emission amount about the raw material in connection with the production of the target product of the target type,
the main facility unit emission amount calculation step includes calculating, for each of a plurality of main facilities respectively associated with the steps in the production of the target product of the target type, a second unit CO₂ emission amount per unit about each of the main facilities on the basis of a total production load amount required in the main facility associated with the step to execute the step in the production in the predetermined period and a total CO₂ emission amount about the main facility with respect to the total production load amount, and
the step emission amount calculation step includes calculating, for each of the main facilities respectively associated with the steps in the production of the target product of the target type, a step CO₂ emission amount about the step executed by the main facility in connection with the production of the target product of the target type, on the basis of the second unit CO₂ emission amount per unit about the main facility calculated in the main facility unit emission amount calculation step and the total production load amount required in the main facility in the production in the predetermined period in connection with the production of the target product of the target type.
